(19) United States
(12) Patent Application Publication
(10) Pub. No.: US 2023/0377748 A1
(43) Pub. Date: Nov. 23, 2023

(54) A Neural Graph Model for Automated Clinical Assessment Generation

(71) Applicant: University of Massachusetts, Boston, MA (US)

(72) Inventors: Zhichao Yang, Amherst, MA (US); Hong Yu, Shrewsbury, MA (US)

(21) Appl. No.: 18/246,892

(22) PCT Filed: Oct. 1, 2021
(86) PCT No.: PCT/US2021/053104

§ 371 Date: Mar. 28, 2023

Related U.S. Application Data
(60) Provisional application No. 63/086,528, filed on Oct. 1, 2020.

(51) Int. Cl. G16H 50/20; G16H 10/60; G16H 50/70; G06F 40/40

(52) U.S. Cl. CPC G16H 50/20; G16H 10/60; G16H 50/70; G06F 40/40

(57) ABSTRACT

Embodiments generate medical support text, e.g., assessments and plans, based on patient medical data. One such embodiment begins by receiving medical data for a given patient. Next, a patient knowledge graph for the given patient is generated based on the received medical data and an expanded graph is generated by expanding the patient knowledge graph based upon supplementary data. In turn, the medical support text for the given patient is generated based upon the expanded graph.

### GOVERNMENT SUPPORT

This invention was made with government support under contract number R01HL125089 from the National Institutes of Health. The government has certain rights in the invention.

### RELATED APPLICATION

This application claims the benefit of U.S. Provisional Application No. 63/086,528, filed on Oct. 1, 2020. The entire teachings of the above application are incorporated herein by reference.

### BACKGROUND

Electronic health records (EHRs) are used by hospitals in the United States and other countries, resulting in an unprecedented amount of digital data or EHRs associated with patient encounters. Rich clinical information is documented in the EHRs. As such, in recent years, secondary use of EHRs has helped advance EHR-related computational approaches to foster precision medicine and a learning health system.

### SUMMARY

While rich clinical information is documented in EHRs, and EHR-related computational approaches have been developed, improved functionality is needed to better utilize the wealth of information that is available from EHRs.

One of the fundamental goals of artificial intelligence is to build computer-based expert systems. Inferring clinical diagnoses to generate a clinical assessment and/or plan during a patient encounter is a crucial step towards building a medical diagnostic system. Previous works were mainly based on either medical domain specific knowledge, or patients' prior diagnoses and clinical encounters.

Embodiments go further and implement an innovative graph neural network to leverage the information in EHRs to generate medical support text, e.g., a medical assessment or plan. An embodiment utilizes a new model for an automated clinical assessment generation. Embodiments implement and employ an innovative graph neural network, where rich clinical knowledge is incorporated into an end-to-end corpus-learning system.

One such embodiment, treats assessment generation as a concept-to-text generation problem. First, such an embodiment generates a local or patient-specific concept graph by natural language processing the free text of the subjective and objective sections of an EHR. The patient-specific concept graph is then expanded with background knowledge extracted from an external and comprehensive knowledge resource, such as the Unified Medical Language System (UMLS). Once the concept-graph is developed, an embodiment, which is trained end-to-end, generates the medical support text from the expanded concept graph.

Embodiments may normalize concepts. For example, “MI”, “myocardial infarction,” and “heart attack” can be mapped in graphs utilized by embodiments to the same concept to mitigate out of-vocabulary errors. In embodiments, the patient-specific concept graph helps generate the reasons for the diagnosis, and the expanded concept graph with the background knowledge helps infer novel text (diagnosis, assessment, and plan text) not described in the input text (e.g., chief complaint, subjective, and/or objective sections of the EHR).

Another embodiment is directed to a method for generating medical support text based on patient medical data. The method begins by receiving medical data for a given patient and, in response, generating a patient knowledge graph for the given patient, based on the received medical data. Next, the method generates an expanded graph by expanding the patient knowledge graph based upon supplementary data. In turn, medical support text for the given patient is generated based upon the expanded graph.

According to an embodiment, the received medical data includes text describing medical symptoms for the given patient. According to an embodiment, the received medical data is patient medical data in a text format. In an embodiment, receiving the medical data comprises accessing an EHR database and obtaining an EHR for the given patient from the accessed database, wherein the obtained EHR comprises the medical data for the given patient. According to an embodiment, the obtained EHR is structured to include: a chief complaint, subjective data regarding the given patient, objective data regarding the given patient, an assessment of the given patient, and a treatment plan for the given patient.

An embodiment generates the patient knowledge graph based on the received medical data by natural language processing the received medical data. In an embodiment, natural language processing the received medical data includes extracting concept-relation-concept triples from the data.

According to an embodiment, the generated patient knowledge graph is a graph indicating relations between concepts in the received medical data. Similarly, in another embodiment, the supplementary data used for the expanding is a concept graph. According to another embodiment, expanding the generated patient knowledge graph based upon the supplementary data comprises computing a graph union of the patient knowledge graph and the concept graph. In such an embodiment the graph union is the expanded graph. Further, according to another embodiment, the concept graph is an external medical knowledge concept graph.

Various techniques may be implemented in embodiments to expand the knowledge graph. For example, in an embodiment, the expansion is implemented by performing a maximum inner product search (MIPS) of a patient database to identify one or more patients similar to the given patient. In turn, medical data for the identified one or more patients similar to the given patient is obtained and the generated patient knowledge graph is expanded using the obtained medical data for the identified one or more patients similar to the given patient. In such an embodiment, the obtained medical data for the identified one or more patients may include at least one of: diagnoses ICD codes for the identified one or more patients; assessments for the identified one or more patients; and treatment plans for the identified one or more patients.

In another embodiment, the patient knowledge graph is expanded by first obtaining lab, diagnosis, and medication codes for one or more previous medical appointments for the given patient. Next, at least one of a medication code and a diagnosis code for a future medical appointment for the given patient are predicted based on the obtained lab, diagnosis, and medication codes. In turn, the generated patient knowledge graph is expanded based upon the predicted at least one medication code and diagnosis code for the future medical appointment.

According to an embodiment, the generated support text is natural language text. Moreover, in an embodiment, the generated support text is a medical assessment for the given patient. Further, in yet another embodiment, the generated support text is a treatment plan for the given patient.

Another embodiment is directed to a computer system for generating medical support text. The system includes a processor and a memory with computer code instructions stored thereon. In such an embodiment, the processor and the memory, with the computer code instructions, are configured to cause the system to implement any embodiments or combination of embodiments described herein.

Yet another embodiment is directed to a computer program product implementation. The computer program product includes a non-transitory computer-readable medium with computer-readable program instructions stored thereon. The instructions, when executed by a processor, cause the processor to implement any embodiments or combination of embodiments described herein.

It is noted that while embodiments are described herein as being implemented to generate medical support text, embodiments are not so limited and may be employed to generate text in any field. In particular, embodiments can be applied in any domain to go from text to graph to text. For example, embodiments can be applied to dialogue systems. In such implementations embodiments could be used to implement an automated teacher assistant that infers a new question for a student or an automated phone service that helps find the best department for a user based on the user's question. Embodiments can also be employed to implement a system that infers a future event based on the current facts (e.g., if we know a series of physical events (weather related, geological, spread of disease, plight, famine, etc.), what would be a future event/effect or state in the series of physical events).

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 depicts an electronic health record that may be utilized in embodiments.

FIG. 2 is a flow diagram of a method for generating medical support text according to an embodiment.

FIG. 3 is a simplified depiction of a concept graph and relations therein that may be generated in embodiments.

FIG. 4 is a schematic diagram of a graph to text framework in which embodiments may be implemented.

FIG. 5 is a schematic diagram of an encoder that may be employed in embodiments.

FIG. 6 is a schematic diagram of a decoder that may be employed in embodiments.

FIG. 7 is a schematic view of a computer network environment in which embodiments may be deployed.

FIG. 8 is a block diagram of a computer node in the network of FIG. 7.

### DETAILED DESCRIPTION

A description of example embodiments follows.

As noted above, electronic health records (EHR) are widely used by hospitals and care providers in the United States and other countries. As a result, there is an unprecedented amount of digital medical data regarding patient encounters and medical treatment. In recent years, secondary use of this data has helped advance EHR-related computational approaches to foster precision medicine and a learning health system (Evans, 2017).

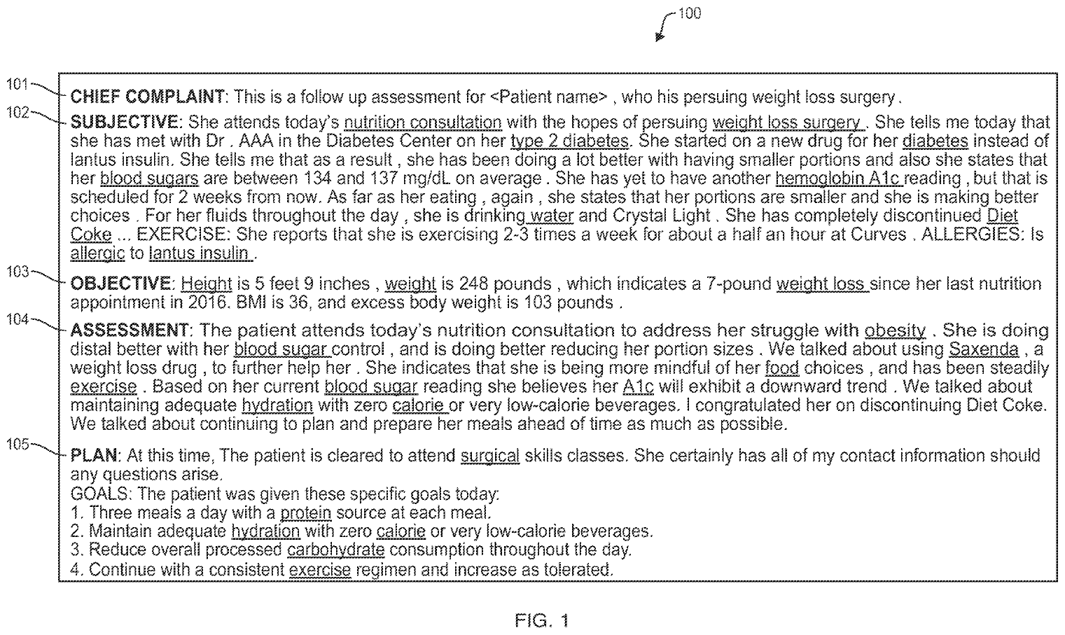

Rich clinical information is documented in the EHRs. Among many structures and formats in EHRs, a problem-oriented SOAP (Subjective, Objective, Assessment, and Plan) structure is commonly used by providers (Podder et al., 2020). FIG. 1 illustrates an example of a SOAP note 100 for an outpatient encounter. The note 100 includes a chief complaint section 101, a subjective section 102, and objective section 103. The note 100 also includes an assessment section 104 and plan section 105.

Typically, chief complaint 101 includes a brief description of a patient's conditions and the reasons for the visit. The subjective section 102 is a detailed report of the patient's current conditions, such as source, onset, and duration of symptoms, mainly based on the patient's self-reporting. The subjective section 102 also usually includes a history of present illness and symptoms, current medications, and allergies. The objective section 103 documents results of physical exam findings, laboratory data, vital signs, and descriptions of imaging results. The assessment section 104 typically contains medical diagnoses and reasons that lead to medical diagnoses. The assessment section 104 is typically based on the content from the chief complaint 101, the subjective section 102, and the objective section 103. The plan section 105 addresses treatment plans based on the assessment 104.

Inferring clinical diagnosis to generate an assessment and plan is a crucial step during doctor-patient encounters. Attempts have been made to automatically determine assessments and plans, but these attempts have been inadequate.

Earlier systems are mainly knowledge based and typically rely upon decision rules. Machine learning approaches have been developed, mainly using longitudinal electronic health records to predict ICD codes (Subotin and Davis, 2014; Amoia et al., 2018), the diagnostic codes assigned to EHRs after each patient's visit or encounter. However, ICD codes are used mainly for billing purposes and have limitations (e.g., incomplete assignment) when used as diagnoses labels (O'malley et al., 2005).

Embodiments propose an alternative task. Instead of predicting ICD codes, embodiments build an expert system by directly generating medical assessments and plans. Embodiments accomplish the task of automated assessment and plan text generation using supervised machine-learning. Specifically, an embodiment system's input is the free-text of an EHR, e.g., chief complaint section 101, subjective section 102, and objective section 103, and the output is the assessment text 104 and/or plan text 105. One such embodiment relies on a supervised machine learning model that is trained based on the SOAP-structured EHR notes as a text to text generation natural language processing (NLP) application. Embodiments solve numerous challenges to provide this text to text generation, including: (i) the variable length of assessments and (ii) the verbose nature of the subjective and objective sections of EHRs.

The length of assessments in EHRs varies, from being short to being verbose. This is because the assessment is mainly inferred (not a mere summary) from the corresponding subjective and objective sections, and the assessment also includes reasons for diagnoses and, thus, the overlap between the input and output word tokens is small. EHR data used in embodiments shows that there is only 12.8% word overlap between subjective and objective sections and the corresponding assessments. This makes the text generation a challenging NLP task. In addition to both the subjective and objective sections being verbose, these sections contain abundant medical jargon, many of which are sparse (with low term frequency) and therefore, are susceptible to being be considered out-of-vocabulary words to the artificial intelligence or expert system implementing the text generation.

A strong baseline model for automated assessment generation is a Pointer-Generator model N2MAG (Hu et al., 2020). Although the model helps mitigate the out-of-vocabulary challenge, it does not address the challenge of limited word overlap between the subjective and objective sections and the assessment and plan.

Therefore, embodiments propose a new method for automated clinical assessment generation, which generates a patient assessment and plan using a knowledge graph. One such embodiment treats assessment/plan generation as a concept-to-text generation problem.

An example embodiment first builds a local or patient-specific concept graph by NLP-processing the free text of the subjective and objective sections. This patient-specific concept graph is then expanded with background knowledge extracted from an external and comprehensive knowledge resource, such as the Unified Medical Language System (UMLS) (Bodenreider, 2004). Once the concept-graph is built, the method generates the assessment and plan from the built concept graph. This assessment and plan text generation is performed by a model, e.g., a decoder, that is trained end-to-end as further detailed below.

Embodiments mitigate both challenges mentioned above. First, the embodiments described herein provide concept normalization to mitigate the out-of-vocabulary word challenge. Further, the patient-specific concept graph generated by the embodiments helps generate the reasons for the diagnosis, and the expanded concept graph with the background knowledge helps infer novel text (diagnosis) not described in the input text (i.e., chief complaint, subjective and objective sections).

Embodiments are built on an innovative graph neural network, where rich clinical knowledge is incorporated into an end-to-end corpus-learning system. The system (embodiments) learn to generate assessment and plan text from objective and subjective sections of a medical graph. The system generates novel content for the assessment text including differential diagnoses or other important related discussions that do not appear in the input text. The new functionality described herein generates the medical support text, i.e., assessment and plan, using a knowledge graph.

Advantageously, embodiments provide a novel method of using a knowledge-graph to generate EHR texts. Further, the knowledge graph of embodiments incorporates not only the local or patient specific concept relations extracted directly from EHR notes, but also rich background knowledge from other sources of relevant data, e.g., an external medical knowledge graph. Moreover, extensive experiments described hereinbelow, show that both the graph neural network architecture and expanded medical background information graph of embodiments improves accuracy of the generated assessment and plan.

### Existing Work

### Text Generation In EHR

Motivated by sharing EHR note data without compromising patient privacy information, a body of work in EHR-related text generation focused on generating synthetic EHR notes. However, most of this work uses discrete features or text data as input while, in comparison, embodiments use a graph of discrete features connected together with relations. Choi et al. (2017) proposed generating synthetic patient records using a combination of an autoencoder and generative adversarial networks (GAN). However, this Choi method only generates high-dimensional discrete variables (e.g., diagnosis, medication, or procedure codes) that act as patient records for secondary analysis instead of free text. Lee (2018) developed an encoder-decoder framework where the encoder's input consisted of numerous discrete variables (e.g., age and ICD codes), and the output of the decoder was chief complaint text. Guan et al. (2018) used the same GAN framework to generate the chief complaint using its EHR note text as the input, but not the structured graph data formats that embodiments utilize. While most previous works generated short EHR text (usually less than 30 words) from either discrete variables or free text, embodiments target a novel task: generating document-wise text from a medical graph that is generated based on the text of medical record(s).

Another existing work, Hu et al. (2020), proposed an augmented attention-over-attention pointer-generator network to summarize the content from the subjective and objective sections of medical records. However, this summarization approach generates short and concise summaries. While the diagnosis information can be copied and pasted from the input text, the Hu model is limited in its ability to generate novel content, which in embodiments, include differential diagnoses or other important related discussions that do not appear in the input text.

### Structured Data To Text

Wiseman et al. (2017) studied the challenges of applying neural networks to the data-to-text task. Wiseman introduced a large-scale dataset where a text review of a basketball game is paired with tables of team and player statistics (points, field goals, rebounds, etc.). However, these tasks focused on text generation from tables, where relation information is not included.

Due to the success of transformer models in applications such as machine translation and graph neural networks, there has been a recent trend to generate longer text (such as paragraph-length text) from structured data. One such example is Koncel-Kedziorski et al., 2019, which introduced a graph to text task by collecting 40,000 Semantic Scholar Corpus taken from the proceedings of AI conferences. Given a knowledge graph constructed by an automatic information extraction system and a scientific article's title, the goal of Koncel-Kedziorski is to generate a corresponding abstract. However, the Koncel-Kedziorski graph only captures relevant information parallel to the text, but not extra information from the background.

### Methods

Embodiments solve the problems of the aforementioned existing methods and provide novel functionality for generating medical assessments and plans.

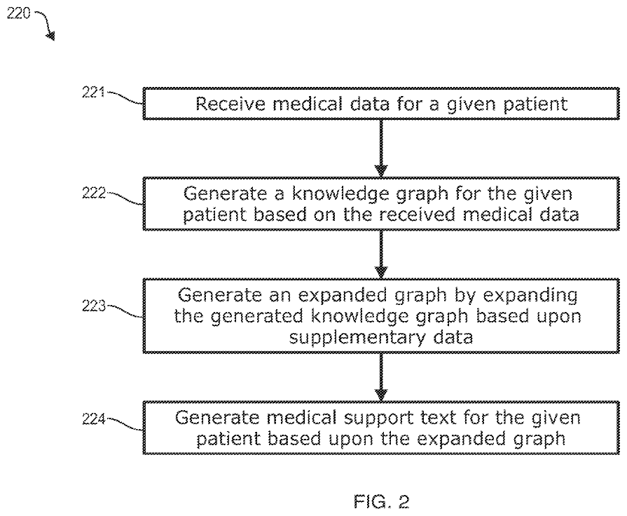

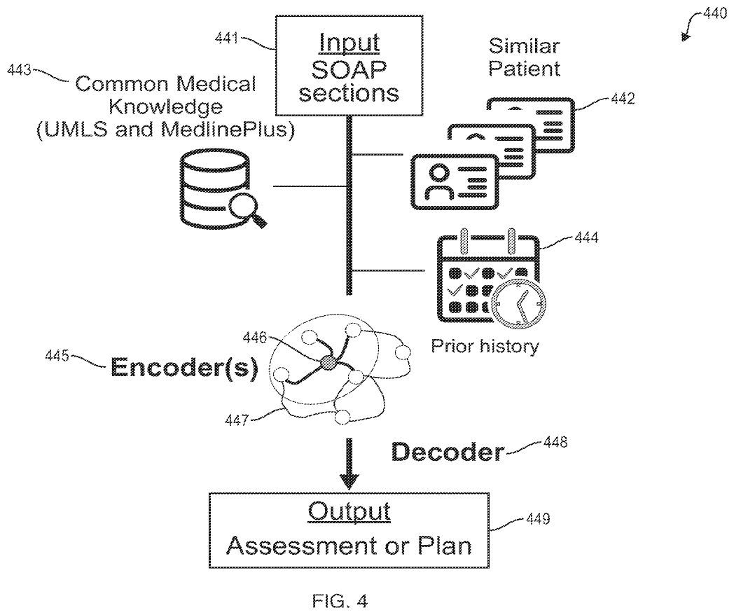

FIG. 2 is a flow chart of one such example method 220. The method 220 is computer implemented and may executed using any computing structure known in the art. Further, embodiments of the method 220 may be implemented in the framework 440 described hereinbelow in relation to FIG. 4.

The method 220 begins at step 221 by receiving medical data for a given patient. The method 220 is computer implemented and, as such, the medical data may be received at step 221 from any storage memory that can be communicatively coupled to a computing device implementing the method 220. For instance, in an embodiment, receiving the medical data 221 comprises the computing device implementing the method 220 accessing an EHR database via a network and obtaining an EHR for the given patient from the accessed EHR database. In such an embodiment, the obtained EHR comprises the medical data for the given patient. In another embodiment, the medical data is received at step 221 via patient or physician input on an online form or interface.

The medical data received at step 221 can be any data related to the given patient and be in any form, e.g., free text. According to an embodiment of the method 220, the medical data received at step 221 includes text describing medical symptoms for the given patient. In another embodiment, the medical data received at step 221 is structured in the same manner as the SOAP note 100 described hereinabove in relation to FIG. 1. In particular, such received medical data includes: (i) a chief complaint, (ii) subjective data regarding the given patient, (iii) objective data regarding the given patient, (iv) an assessment of the given patient, and (v) a treatment plan for the given patient.

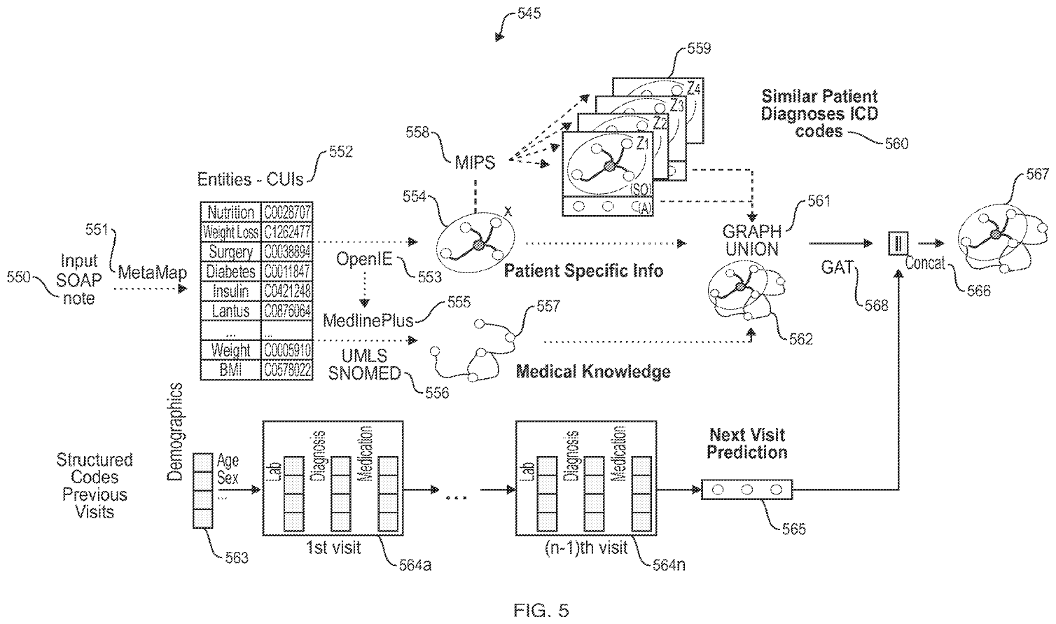

To continue, at step 222, the method 220 generates a patient knowledge graph for the given patient based on the received medical data. According to an embodiment, the patient knowledge graph generated at step 222 is a graph indicating relations between concepts in the medical data received at step 221. An embodiment of the method 220 generates the patient knowledge graph at step 220 by natural language processing the received medical data. According to an embodiment, such natural language processing includes extracting concept-relation-concept triples from the medical data received at step 221. At step 222 in an embodiment where the received medical data is a structured SOAP note (an example of which is shown in FIG. 1), the method 220 generates the patient knowledge graph based on the received medical data by natural language processing both (i) the subjective data and (ii) the objective data. In an embodiment, natural language processing both the subjective and objective data includes extracting concept-relation-concept triples from the subjective data and objective data. In turn, the relationships between these extracted concept-relation-concept triples are embodied in a graph. In an embodiment, at step 222, the method 220 employs the encoders described herein to create the knowledge graph for the patient. For instance, such an embodiment may utilize the encoders 445 and/or 545 described hereinbelow in relations to FIGS. 4 and 5, respectively.

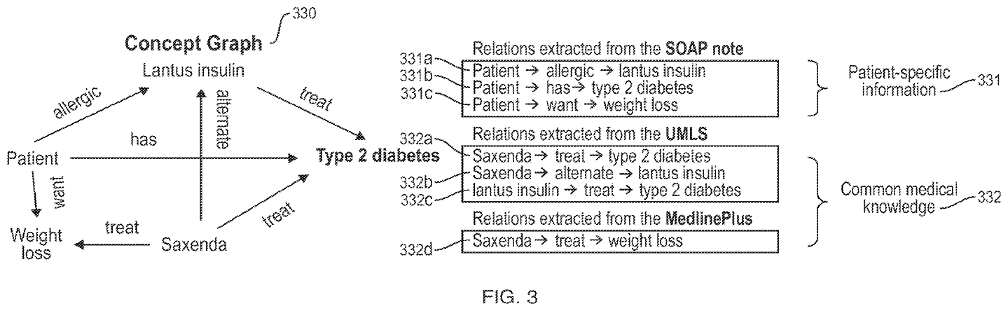

To illustrate the functionality of generating 222 a patient knowledge graph, consider the particular examples in FIGS. 1 and 3. In this illustrative example, the medical data received at step 221 is the SOAP note 100 shown in FIG. 1. At step 222, natural language processing is performed on the soap note 100 and the patient information relations 331 shown in FIG. 3 are extracted. Specifically, the relations 331a “patient→allergic→lantus insulin,” 331b “patient→has→type 2 diabetes,” and 331c “patient→want→weight loss.” Continuing the functionality of step 222, the relations 331a-c are organized into the concept graph 330. It is noted that the concept graph 330 depicted in FIG. 3 is an expanded concept graph that is described in further detail hereinbelow. However, the concept graph generated at step 222 is similar to the graph 330, but in this illustrative example, only embodies the relations 331a-c.

To continue the method 220, at step 223, an expanded graph is generated by expanding the patient knowledge graph based upon supplementary data. In embodiments of the method 220, the supplementary data may be any such additional data that a user wants considered in generating medical support text. For instance, the supplementary data may be data extracted from MedlinePlus and/or UMLS SNOMED. Various techniques may be implemented in embodiments of the method 220 to expand the knowledge graph at step 223.

In an embodiment, the supplementary data is a concept graph. In such an embodiment, expanding 223 the generated patient knowledge graph based upon the supplementary data comprises computing a graph union of the patient knowledge graph generated step 222 and the concept graph supplementary data. In such an embodiment, the graph union is the expanded graph.

In another embodiment, the supplementary data is an external medical knowledge concept graph, i.e., a graph showing the relationships between medical concepts. Such an embodiment expands 223 the graph generated at step 222 with the external medical knowledge concept graph by computing the union of the two graphs, the graph generated at step 222, and the external medical knowledge concept graph.

According to another embodiment, the graph from step 222 is expanded at step 223 by first performing a maximum inner product search (MIPS) of a patient database to identify one or more patients similar to the given patient. It is noted that while step 222 is described as using MIPS as a similarity measure to identify one or more patients similar to the given patient, embodiments are not so limited, and may utilize any similarity measurement techniques known to those of skill in the art. Second, medical data for the identified one or more patients similar to the given patient is obtained. Third, the patient knowledge graph generated at step 222 is expanded at step 223 using the obtained medical data for the identified one or more patients. In such an embodiment, the obtained medical data for the identified one or more patients may include at least one of: diagnoses ICD codes for the identified one or more patients; assessments for the identified one or more patients; and treatment plans for the identified one or more patients.

In another embodiment of the method 220, the patient knowledge graph is expanded at step 223 by first obtaining lab, diagnosis, and medication codes for one or more previous medical appointments for the given patient. Next, at least one of a medication code and a diagnosis code for a future medical appointment for the given patient are predicted based on the obtained lab, diagnosis, and medication codes. This prediction can be performed in a plurality of different ways. For instance, one such embodiment uses a knowledge graph in SNOMED CT. Such an embodiment may determine from the previous medical appointment data that a drug, DrugA, treats a disease. The SNOMED CT graph may show that DrugB also treats the disease. At the current appointment, the patient indicates that she had an allergic reaction to DrugA. Based upon the SNOMED CT graph, such an embodiment can infer that DrugB is going to replace DrugA. Another embodiment makes the prediction by predicting a new node or concept using the functionality described in U.S. Pat. No. 10,628,748. Yet another embodiment uses a pretrained end-to-end model, such as G-BERT. To continue, the patient knowledge graph (from step 222) is expanded based upon the predicted at least one medication code and diagnosis code for the future medical appointment.

An embodiment expands the graph at step 223 using an external general knowledge graph through use of graph addition. In another embodiment, node prediction or link prediction methodologies, such as TransE described at https://ojs.aaai.org/index.php/AAAI/article/view/8870, are used to expand the patient's knowledge graph.

To illustrate the expansion implemented at step 223, once again consider the functionality illustrated in FIG. 3. In this example, the supplementary data used for expansion is the common medical knowledge 332. Here the supplemental data 332 includes the concept relations 332a-c extracted from the UMLS database and the concept relation 332d extracted from the MedlinePlus database. The relevant supplemental data 332 indicates “saxenda→treat→type 2 diabetes” 332a, “saxenda→alternate→lantus insulin” 332b, “lantus insulin→treat→type 2 diabetes” 332c, and “saxenda→treat→weight loss” 332d. These concept relations 332a-d are incorporated into the concept graph 330 which, at step 222, only included the concept relations 331a-c. An embodiment incorporates the concept relations 332a-d into the concept graph 330 by computing a graph union.

In an embodiment, at step 223, the method 220 employs the encoders described herein to create the expanded graph. For instance, such an embodiment may utilize the encoders 445 and/or 545 described hereinbelow in relations to FIGS. 4 and 5, respectively.

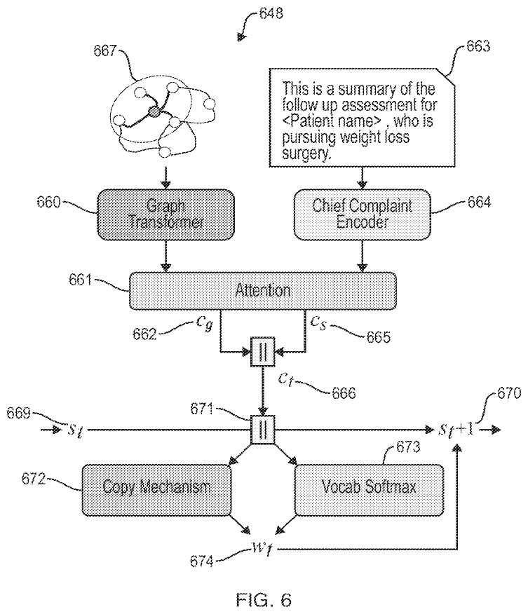

Returning to FIG. 2, the method 220 continues by generating 224 the medical support text for the given patient based upon the expanded graph. According to an embodiment, the generated 224 support text is natural language text. In an embodiment, the generated 224 support text is a medical assessment for the given patient. Further, in yet another embodiment, the generated 224 support text is a treatment plan for the given patient. An embodiment of the method 220 employs the decoders described herein at step 224 to generate the medical support text. For instance, such an embodiment may utilize the decoders 448 and/or 648 described hereinbelow in relation to FIGS. 4 and 6, respectively. The medical support text generated at step 224 can be used for a plurality of different applications. For example, the generated text can be used to provide clinical decision support. In such applications, the text can assist medical providers with diagnosing patients and prescribing treatment plans, e.g., medications. The generated text can also be used by medical providers to identify other care providers, e.g., specialists, to whom to refer patients. Further, the medical support text can provide patient-centric support. The generated text can help patients identify materials that support their goals, e.g., diet products, to help prevent negative clinical outcomes.

Another embodiment implements a new end-to-end model for automated medical assessment and plan generation. Such an embodiment treats assessment and plan generation as a text-graph-text generation problem, which allows knowledge grounding. FIG. 4 shows an example embodiment framework 440 that provides this functionality. FIG. 4 shows an example model according to an embodiment that uses the encoder-decoder framework 440. Instead of a typical text-input encoder for text generation, the framework 440 uses a graph transformer as an encoder 445. In the framework 440, the input is a SOAP note 441 of a patient. In such an embodiment, the encoder 445, i.e., graph transformer, integrates a patient's prior medical history 444, information regarding patients with similar conditions 442, and external medical knowledge 443 with the input sections 441 from an EHR for the patient to create the graph 447 centered around the patient entity 446.

In the framework 440, for a patient visit, the framework 440 first identifies the subjective and objective (SO) sections 441 of the SOAP note. An embodiment uses MetaMap to identify clinical concepts from the SO sections 441, from which Open IE is used (Stanovsky et al., 2018) to generate the patient specific concept graph (not shown). In the framework 440, the patient specific concept graph is augmented with common medical knowledge data 443, e.g., UMLS and MedlinePlus data, using concept relations in SNOMED-CT (Donnelly, 2006). The framework 440 uses Open IE (Stanovsky et al., 2018), to extract a concept graph from the medical knowledge data 443, e.g., MedlinePlus, and then augments the patient's concept graph with the data 443. For potential medical knowledge that is not in the external knowledge resource data 443 (UMLS and MedlinePlus), the framework 440 identifies patients with similar conditions and injects the similar patient data 442 for knowledge inference. Specifically, for a patient specific information 442 graph x at nₜₕ visit, the framework 440 uses Maximum Inner Product Search (MIPS) to find the top-K similar subjective and objective parts from EHR of patient encounter zᵢ and, then, retrieves the assessment/plan sections of each similar patient encounter for the downstream assessment generation/plan of patient visit n.

To incorporate previous patient encounters 444, the framework 440 uses a separate transformer encoder that takes in structured codes from previous visits to predict the assessment/plan code. Specifically, given a patient's demographics information, lab, diagnosis, and medication codes of the previous (n−1) visits, the framework 440 uses a transformer encoder to predict diagnosis and medication codes for the nth visit for the given patient (the patient for which an assessment/plan is being determined). This prediction can be considered as an external concept graph and be incorporated by computing a graph union, or can be incorporated as an additional attention over attention (a model described in Hu et al., 2020).

Through the aforementioned functionality, the framework 440 integrates a patient's prior medical history 444, information from patients with similar conditions 442, and external medical knowledge 443 with sections 441 from an EHR for the patient, to form the graph 447 centered around the patient entity 446. This graph 447 is encoded by the encoder 445 and passed to the decoder 448. In an embodiment, the encoder 445 is a model that learns from the input (text or graph), and transforms the input into a representation of the input. An encoder can be used as a classifier to identify whether the input is relevant or not relevant. The decoder 448 can take the encoder 445 representation and output a sequence, including graph or text.

For final assessment or plan prediction y 449, the decoder 448 of the framework 440 treats the combined graphs/codes embedding 447 mentioned above as a latent variable from four different sources, SOAP note sections 441, medical knowledge data 443, similar patient data 442, and the patient's prior medical data 444, and marginalizes over the seq2seq predictions end-to-end to generate the assessment and/or plan 449. Such functionality marginalizes the four different sources of data, SOAP note sections 441, medical knowledge data 443, similar patient data 442, and the patient's prior medical data 444 which may be in disagreement.

EHR Cohorts

Embodiments may utilize, e.g., for training and text generation, data from any desired EHR cohorts. Amongst others, embodiments can utilize the VHA cohort and Pitts cohort.

One such embodiment utilizes longitudinal EHR data from the U.S. Department of Veterans Affairs. The U.S. Department of Veterans Affairs is composed of 1,255 healthcare facilities (sites), including 170 Veterans Affairs Medical Centers, and 1,074 outpatient facilities (noa, 2021). The VHA (Veterans Health Administration) EHR cohort comprises all patients between the ages 18 and 90 who were admitted for secondary care to medical or surgical services from the beginning of October 2015 to the end of September 2019 and for whom there was at least one year of electronic health record data before admission. The VHA EHR cohort has a total of 8,208,742 patients and 306,243,564 clinical visits. An embodiment utilizes note types from the VHA cohort, as shown in Table 1, that have the explicit SOAP structure. These notes are included in the VHA cohort, which is composed of a total of 3,273,627 notes (from a total of 1,658,035 patients) with the SOAP structure.

The VHA cohort also includes structured data, such as the International Statistical Classification of Diseases (ICD) codes, Current Procedural Terminology (CPT) codes, laboratory results (including—but not limited to—biochemistry, hematology, cytology, toxicology, microbiology, and histopathology), medications and prescriptions, orders, vital signs, and health factors. In an implementation of an embodiment, data access to the cohort data and computing is conducted using the secure workspace at the VA Informatics and Computing Infrastructure (VINCI).

Another cohort that can be employed in embodiments is the Pitts cohort. This Pitts cohort which can be made available, via a MOU agreement with the University of Pittsburgh BLULab, includes a total of 7,839 and 6,718 de-identified discharge summaries and progress notes from patients receiving care at the University of Pittsburg hospital system between years 2007 and 2008. An embodiment, is trained on the VHA cohort and evaluated using the Pitts cohort. Such functionality may entail training using the SOAP structured EHR notes from the VHA cohort.

Table 1 provides a summary of the VHA and Pitts cohort data and notes the note types, numbers of total notes, and notes with the SOAP structure.

TABLE 1

| Note Type | Total | SOAP | Percentage |
|---|---|---|---|
| primary care note | 60,257 | 35,694 | 59.24% |
| primary care urgent care note | 1,662,418 | 904,401 | 54.40% |
| primary care outpatient note | 849,378 | 234,324 | 27.59% |
| nutrition dietetics | 12,464 | 7,395 | 59.33% |
| nutrition note | 24,139 | 13,790 | 57.13% |
| progress note | 139,966 | 92,693 | 66.23% |
| discharge summary | 344,974 | 58,372 | 16.92% |
| mental health note | 539,731 | 227,590 | 42.17% |
| psychiatry note | 687,652 | 440,869 | 64.11% |
| dentistry note | 2,499,138 | 1,217,933 | 48.73% |
| optometry note | 190,726 | 40,566 | 21.27% |
| total | 7,010,843 | 3,273,627 | 46.69% |

### Concept Graph

Embodiments generate and employ a concept graph for assessment and plan generation. In an embodiment, the concept graph is generated based on text extracted from a medical record, e.g., a SOAP note, and augmented with external medical knowledge.

One such embodiment, builds a concept graph used later for assessment generation by first building a local or patient specific information graph. An embodiment first builds a local or patient specific concept graph by NLP processing the free text of the SOAP note. An example embodiment performs the NLP processing on the subjective and objective sections to create a graph for generating an assessment, and performs the NLP processing on the subjective, objective, and assessment sections to create a graph for generating a plan. This patient-specific concept graph is then expanded with external medical knowledge extracted from resources for external medical knowledge. What follows is first a description of resources for external medical knowledge and then a description of how to create the concept graph for embodiments.

### Resources For External Medical Knowledge

The Unified Medical Language System (UMLS) (Boden-reider, 2004) is the largest biomedical resource managed by the U.S. National Library of Medicine (NLM). It is a set of files and software that brings together health and biomedical vocabularies to enable interoperability between computer systems. The UMLS includes the Metathesaurus, a large biomedical thesaurus organized by concept and concept relations from nearly 200 different vocabularies. The UMLS incorporates the Systematized Nomenclature of Medicine Reference Terminology and Clinical Terms Version (SNOMED-CT) (Donnelly, 2006), the most comprehensive collection of clinical terminology comprising 340,000 concepts and their relations. MetaMap (Aronson, 2001) is a tool, also developed by NLM, that maps biomedical text to the UMLS Metathesaurus. An embodiment uses MetaMap to identify clinical concepts from EHR notes, and then forms the concept graph with their corresponding concept relations in the SNOMED-CT. For example, the underlined terms in the SOAP note 101 of FIG. 1 can be mapped to the UMLS concepts, and their concept relations form the concept graph generated at step 222 of the method 220 described hereinabove in relation to FIG. 2.

The UMLS Metathesaurus is organized by a semantic network, comprising 127 semantic types and 54 semantic relations between semantic types. Each Metathesaurus concept is assigned at least one semantic type. Embodiments can also include concept relations between semantic types.

MedlinePlus (Miller et al., 2000), also developed by NLM, is a collection of high quality health information. Medline Plus describes symptoms, causes, treatments, and prevention information of over 1000 diseases, illnesses, health conditions, and wellness issues. Embodiments can use Open IE (Stanovsky et al., 2018) to extract external medical knowledge from MedlinePlus to augment, i.e., expand, a patient's concept graph. For example, MedlinePlus includes the description that “Liraglutide injection (Saxenda) is used along with a reduced calorie diet and exercise plan to help people who are obese or who are overweight and have weight related medical problems to lose weight and to keep from gaining back that weight.” This description can be used to extract a relation, i.e., the relation 332d, between Saxenda and weight loss and this relation 332d can then be used to augment patient's concept graph.

### Concept Identification and Concept Graph

To build a concept graph, for instance at step 222 of FIG. 2, an embodiment first builds a patient specific information graph by extracting concept-relation-concept triples from text in the subjective and objective sections for assessment and in the subjective, objective, and assessment sections of the patient's EHR for generating a plan.

An embodiment uses OPENIE (Stanovsky et al., 2018) to extract triples, each of which consists of a subject (usually the patient), object, and the open domain relation between the subject and object specified in the text. In embodiments, the patient specific information graph typically shares most of the patient's key clinical information stated in the subjective and objective sections of each EHR, including past diagnosis, symptoms, current medications, and allergies, amongst other information.

An embodiment also implements functionality to increase word overlap between the subjective and objective sections and the assessment section. To do so, an embodiment applies the Unified Medical Language System (UMLS) (Bodenreider, 2004) to build a background medical knowledge graph. The UMLS includes a large biomedical thesaurus that is organized by concept (meaning) and concept relations from nearly 200 different professional medical vocabularies. This functionality allows nodes, like symptoms, diagnosis, and treatment, to be linked together, which constitute the relevant background knowledge for a patient.

Before building a medical concept graph for each EHR, an embodiment first extracts all medical relevant entities as key clinical info. One such embodiment uses MetaMap (Aronson and Lang, 2010) to identify all key medical phrases and maps the identified key medical phrases to certain medical concepts, referred to as Concept Unique Identifiers (CUIs) in the Unified Medical Language System. Such an embodiment may employ such functionality to reduce the variations in natural language and to ensure the accuracy of clinical meanings captured by the triples extracted from the EHR.

One such implementation maps the free text to the UMLS concepts (CUIs) and then identifies the relation between the concepts. For example, “MI,” “myocardial infarction” and “heart attack” can now be mapped to the same concept. This mapping also mitigates the out-of-vocabulary word (e.g., MI) challenge. An embodiment uses MetaMap (Aronson, 2001) to identify all UMLS concepts. The patient's concept graph can then be augmented with concept relations extracted from the UMLS and the MedlinePlus databases. One such embodiment uses Open IE (Stanovsky et al., 2018) to extract concept relation triples from MedlinePlus. The use of MetaMap allows such an embodiment to associate extracted lexicons with their conceptual semantics, since words and phrases will be mapped to the same CUIs if they are semantically equivalent.

To build a patient specific information graph Gₛ, an embodiment uses OPENIE (Stanovsky et al., 2018) to extract all relevant relations mentioned in the text (the patient's EHR). In an implementation, only triples where CUIs exist are included because they represent key clinical info with respect to the specific patient. Since sentences from EHR text are not necessarily written in a grammatical manner, or with clear subject-predicate-object structure, an embodiment may rely on matching rules to identify spans of text corresponding to the symptomatic and other personal information of each patient (gender, age, etc.). In an example embodiment, the matching rules indicate the subject is the “who” or “what” of the sentence, the predicate is the verb, and the object is any noun or concept that is part of the action of the subject.

To build a background medical knowledge graph G_{b}, an embodiment uses the UMLS SNOMED Clinical Terms Database (Bodenreider, 2004) to search for all potential connections between every pair of CUIs, i.e., concepts. If a 1-hop connection is found (i.e., two concepts or nodes in a graph are connected directly), it is included in both the new entity and relations to the graph.

To continue, such an embodiment combines nodes and relations from both the background medical Knowledge graph Gb and patient specific information graph Gₛ, into a combined information graph G, by computing the graph union (G=G_{b}∪U Gₛ).

### Similar Patient Profile

A successful framework for knowledge based expert systems is Case Based Reasoning (CBR) (Chattopadhyay et al., 2013). By utilizing case based reasoning, embodiments can recapitulate previous assessments or plans of similar patients to infer the diagnosis or treatment of a new case. CBR mimics the way doctors make a diagnosis. Given a new patient, CBR's accuracy in practice depends on successful retrieval of similar cases. An embodiment learns a better graph representation of each patient and builds a concept graph by learning from similar patients. In contrast to models that store knowledge in their parameters, this approach explicitly exposes the role of medical knowledge by asking the model to decide what knowledge to retrieve and use during inference. Before making each prediction, the language model uses a retriever (i.e., an element configured to perform information retrieval) to retrieve similar patient data based on their labs and symptoms from a data cohort, e.g., the VHA cohort, and then uses those similar patients' corresponding assessments or plans to help inform a prediction.

In order to introduce similar patient assessment or plan data, an embodiment employs a retrieval augmented generation method that combines both pretrained parametric memory (such as parameters in GAT as knowledge) and non-parametric memory (such as medical knowledge graph, similar patient as knowledge) for language generation. An embodiment can use the input graph Gₛ to retrieve similar patient encounter graphs z and can use the retrieved graphs z as additional context when generating the target sequence y (i.e., the assessment or plan being generated).

As shown in FIG. 4, an embodiment can combine two components: (i) an encoder 445 and a decoder 448. In an embodiment, the encoder, e.g., encoder 445, is configured with parameters that returns (top-K truncated) similar patient's encounter graphs given a query Gₛ. In an embodiment, the decoder, e.g., the decoder 448, is parametrized by θ that generates a current token based on a context of the previous i−1 tokens, the original input Gₛ, and a retrieved patient encounter graph z.

To train the encoder and decoder end-to-end, an embodiment treats the retrieved patient specific graph as a part of a latent variable that is marginalized to get the seq2seq probability P(y|Gₛ) via a top-K approximation as shown in Equation 12. This decoder will be discussed in-depth in the later section.

In order to find the potential candidate patient encounters who have similar symptoms and lab results as indicated in the subjective and objective sections of the EHR, e.g., the data 442, the encoder P₆₅ (z|Gₛ) ranks patient encounters by relevancy where a higher probability score represents a higher relevancy between a potential candidate patient encounter and original query patient encounter. This encoder will follow a siamese bi-encoder architecture as shown in Equation 1:

P_{γ}(z|Gₛ) ∝exp(d(z)^{T}q(Gₛ)) d(z)=GAT(z) q(Gₛ)=GAT(Gₛ) (1)

where d(z) is a dense representation of potential relevant patient encounter produced by a Graph Attention Neural Network (GAT) graph encoder, and q(Gs) is the original patient graph representation produced by a query encoder, also based on GAT. Calculating top-k(P_{γ}(*|Gₛ)), the list of k patient encounter z with highest prior probability P_{γ}(z Gs), is a maximum inner product search (MIPS) problem, which can be approximately solved in sub-linear time.

To get the complete representation of the patient, e.g., generate the patient graph at step 222 and expand the patient graph at step 223, an embodiment combines nodes and relations from 3 sources: the background external medical concept graph G_{b}, patient specific information graph Gₛ, and retrieved relevant patient graph G_{z}, into a combined information graph G, by computing the graph union (G=G_{b} ∪Gₛ∪G_{z}). An embodiment, may also consider the patient's prior medical history (444) and incorporate this data into the graph G. After obtaining a complete graph G, an embodiment uses GAT to encode this graph G into high dimension embedding c_{g} that represents contextual information stored in all nodes and relations from the graph G.

As for the decoder text-generation part, the decoder can be modelled using any pre-existing NLP encoder-decoder architecture. The decoder functionality is discussed in detail below.

### Incorporating Longitudinal Information

A patient's prior medical history is important for his current or future clinical profile. A patient's prior medical history is described in the patient's longitudinal EHRs (EHRs from previous visits). While such EHRs can be simply appended to each other sequentially and inputted to pretrained language models, such an approach would result in a longer text length that may exceed the limit of those pretrained language models. Hence, a more memory efficient method is needed to represent each patient encounter. Embodiments provide such functionality.

There has been a substantial amount of research in predictive modeling using longitudinal EHRs. Using a reverse time attention mechanism and a recurrent neural network, RETAIN (Choi et al., 2016) built an interpretable predictive model for heart failure using structured longitudinal data. Patient2Vec (Zhang et al., 2018a) added self-attention within each clinical visit and between visits. This led to the longitudinal representation for each patient. BEHRT (Li et al., 2020) developed pretrained models to predict disease based on the BERT's masked language model. These pretrained models use positional embeddings to represent longitudinal visits. Recently, BART (Lewis et al., 2020) was introduced for text generation applications. BART's autoregressive decoder has shown significant improvements in NLP applications such as machine translation, summarization, and QA. Due to its text generation advance, embodiments can be based on the BART model, and use the BERT model as a strong baseline model. Such an embodiment may use the BART functionality to predict future diagnoses from past EHRs. Further, it is noted that embodiments are not limited to utilizing the BART functionality and embodiments may utilize other encoders and decoders.

To predict future clinical diagnoses from a patient's previous visits, an embodiment first obtains clinical structured data such as abnormal labs l, diagnosis d, and medication m, from the EHR records for these previous visits. This clinical structured data, l, d, and m can be represented as a sequence of multivariate observations for the patient: X={x₁, x₂, . . . , xₜ} where t is the number of previous visits for the patient. Here, to better represent each patient's visits xₜ, an embodiment uses three main structured codes xₜ=lₜ∪dₜ∪mₜ, where the patient's abnormal lab list lₜ, diagnosis list dₜ, and medication list mₜ is a complete dictionary set of lab codes L, diagnosis codes D, medication codes M, respectively. In order to better predict the diagnosis code for later visits, an embodiment builds a new pretrained code model, which focuses on this code prediction problem and this model is used to predict future diagnosis a patient will have.

Given a patient's history X_{1:(t-1)} and lab codes at visit t, such a model is trained to predict multiple diagnosis and medications by generating a multi-label output y^{t}∈0.1^{(D∪M)}. This problem is similar to a self-supervised pretraining objective in the NLP domain where word tokens can be replaced by the structured codes in embodiments described herein. One such example embodiment uses a BART (Lewis et al., 2020) based model to solve this problem.

Similar to self-supervised tasks where the BART (Lewis et al., 2020) model is trained by corrupting tokens in a document, an embodiment is trained by corrupting codes in a patient's record then optimizing a reconstruction loss: the cross-entropy between the decoder's output and the original visit. Since this approach can be considered as an enhancement for assessment or plan generation, an embodiment may only mask diagnosis and medication codes to recover later, because lab codes do not usually occur in assessment or plan section of EHR. Similar to BART (Lewis et al., 2020), an embodiment utilizes a multi-layer transformer architecture. The model takes the masked code as input and derives a code embedding for the last visit:

cₜ=transformer(x₁,x₂, . . . ,xₜ₋₁) (2)

where transformer is a multiple layer encoder decoder model with parameters to be trained. Thus, for the self-prediction task, according to an embodiment, the code embedding cₜ can be learned from random initialization and then by minimizing the loss below:

L=−logP (dₜ|hₜ) −logP (mₜ|hₜ) (3)

By minimizing the loss, an embodiment can pretrain a large code model which has the ability to infer diagnosis and recommended drug code for future patient visits based on the codes from the patient's previous visits. These inferred, i.e., predicted, diagnosis and drug codes can then be used to augment the patient's concept graph, e.g., at step 223.

### Graph To Text

After generating the graph G, the graph G is then used to generate the assessment or plan. One such embodiment first applies a graph neural network to the knowledge graph G using an encoder-decoder framework, e.g., the encoder-decoder framework 440 shown in FIG. 3. In an embodiment, given a knowledge graph constructed by an automatic information extraction system as described above and, optionally the patient's chief complaint, the framework is used to generate a corresponding assessment or plan in text.

### Encoder—Graph Attention Neural Network

This section provides an in-depth description of a Graph Attention Neural Network (GAT) that may be employed in embodiments and provides a description of how embodiments can modify an adjacency matric to find potential relations between two entities.

To encode the graph G (the graph described above that is based upon the data 441, 442, 443, and 444), an embodiment uses GAT. First, such an embodiment obtains an embedding matrix. This may be done using the last hidden state of a bidirectional recurrent neural network (RNN) run over embeddings of each word in the entity phrase to associate a node (mostly multiple words in a medical phrase) to the graph with a continuous representation. Such an implementation may use RNN to represent an entity using a vector representation with a plurality of dimensions, e.g., 200.

[symbol]

The output of this embedding step is a matrix H⁰={h₀⁰,h₁⁰, . . . h_{N}⁰}∈ [symbol] ^{D}, (where N is the number of nodes in the graph G and D is the number of features in each node) which will serve as input (layer 0) to the graph transformer model. The layer then produces a new set of node features H¹={h₀¹,h₁¹, . . . h_{N}¹},h₁¹∈ [symbol] ^{D}, as its first layer output. This step can be repeated for multiple layers to embed the graph extensively.

In order to better encode the input features into next-level features, an embodiment uses extra parameters. First, a linear transformation is carried out using two weight matrices, W_{Q}∈ [symbol] ^{D′×D} to obtain a query matrix, and W_{K}∈ [symbol] ^{D′×D} to obtain a key matrix. Then, such an embodiment performs a self-attention to compute attention coefficients which indicate the importance of node j's features to node i.

e(hᵢ,hⱼ)=(W_{Q}hᵢ)^{T}W_{K}hⱼ (4)

To continue, in order to match attention weights to a probability from 0 to 1, a softmax operation is used to re-scale the importance of all neighboring nodes Nᵢ of node i.

$${α}_{ij} = \frac{exp ( e ( {h}_{i} , {h}_{j} ) )}{{\sum }_{k ∈ {N}_{i}} exp ( e ( {h}_{i} , {h}_{k} ) )} \qquad ( 5 )$$

Once attention weight αᵢⱼ is obtained, the contextualized representation hᵢ of node i is obtained from attending over the connected nodes weighted by attention weight. To stabilize the learning process of self-attention, such an embodiment employs multi-head attention.

$${h}_{i}^{′} = {h}_{i} + {\vert}_{k = 1}^{K} ( {\sum }_{j ∈ {N}_{i}} {α}_{ij}^{k} {W}_{V}^{k} {h}_{j} ) \qquad ( 6 )$$

In equation 6, denotes the concatenation of the K attention heads, Nᵢ denotes in neighborhood of node i, Wᵥ∈ [symbol] ^{D′×D} is used to obtain a value matrix. In such an embodiment, by using concatenating from all heads, the returned output, h′ᵢ, will comprise K×D′ features (rather than D′) for each node. An embodiment uses an adjacency matrix A with weights that has zeros where connections are not allowed, and ones where connections are allowed to represent neighborhood. According to an embodiment neighborhood is an entity and relations centered around an entity, e.g., a concept.

$${h}_{i}^{′} = {h}_{i} + {\vert}_{k = 1}^{K} ( A {α}_{i}^{k} {W}_{V}^{k} h ) \qquad ( 7 )$$

Similar to their work (Vaswani et al., 2017), such an embodiment can use block networks, which consists of a feedforward network with a non-linear transformation and layer normalization, to reduce the dimension back to D′. This stacking method enables information to propagate through the majority of the graph. In an embodiment, blocks are stacked L times to encode information among L hop nodes, with the layernorm output of layer l-1 taken as the input to layer 1. The final output matrix H^{L}={h₀^{L},h₁^{L}, . . . h_{N}^{L}}, hᵢ^{L} ∈ [symbol] ^{D} represents contextual information stored in all nodes and relations from the concept graph, e.g., the graph G generated based on the patient's EHR 441 and expanded based on the data 443, 442, and 444.

In order to find potential relations between two entities, an embodiment relaxes graph connectivity restrictions by allowing more parameter weight updates between different layers in the GAT. Specifically, instead of a strict adjacency matrix A made from a pre-existing knowledge base like SNOMED-CT, an embodiment uses a learnable adjacency matrix with more potential connections allowed and creates a customized mask when calculating attention weights which loosely follow the external knowledge. For example, assume treatment codes are connected to diagnosis codes, but less so to other treatment codes. Based on this assumption, an embodiment can create a customized mask when calculating attention weights that has negative infinities where connections are less likely, and zeros where connections are highly likely.

An embodiment may also encode the chief complaint section using a BiLSTM for chief complaint word embedding P={₀,p₁, . . . ,p|c|} where |c| is the length of a chief complaint sentence. Such an embodiment uses a BiLSTM encoder instead of a graph encoder because chief complaint text is usually concise and each word contains a significant amount of information.

FIG. 5 is a schematic diagram of an encoder 545 that may be employed in embodiments. The encoder 545 takes a SOAP note 550 as input and uses the MetaMap functionality 551 to identify key medical phrases in the SOAP note 550 to the CUIs 552. This mapping between phrases in the soap note 550 and the CUIs 552 is then processed using OpenIE 553 to generate the patient specific graph 554. Further, OpenIE 553 is used to process the MedlinePlus data 555 and UMLS data 556 to generate the medical knowledge graph 557.

To continue, the encoder 545 uses MIPS 558 to find similar subjective and objective sections 559 and similar patient diagnoses ICD codes 560. The data 559 and 560 may be combined into a graph and combined, e.g., via graph union 561 with the graphs 554 and 557 to form the patient graph 562.

The encoder 545 also takes the patient's demographics information 563, lab, diagnosis and medication codes of the previous (n-1) visits 564a and 564n and uses a transformer encoder (not shown) to predict diagnosis and medication codes 565 for the next visit.

The union graph 562 (which may include the graph 560 (graph of similar patient diagnoses ICD codes)) is processed with the GAT network 568 and the results matrix of this processing are concatenated 566 with the predicted data 565 matrix to form the graph 567 that can be processed by the downstream decoder to predict a next word in an assessment or plan.

### Decoder

In order to generate an accurate assessment or plan based on the patient and background information input, an embodiment trains an attention-based decoder with a copy mechanism to extract relevant content from both the knowledge graph and the chief complaint.

At each decoding timestep t, an embodiment uses decoder hidden state st to compute context vectors c_{g} for the graph and context vectors cₛ for the chief complaint sequence.

To compute context vectors c_{g} for the graph, an embodiment uses an approach similar to the approach shown in equations 2 and 3. However, instead of using a specific node as the query to be centered, such an embodiment replaces the specific node with decoder hidden state sₜ of previous timestep t. Moreover, instead of using a neighborhood centered around a node, such an embodiment allows hidden representation from last layer hⱼ^{L} from every node V to attend on query.

$${c}_{g} = {s}_{t} + {‖}_{k = 1}^{K} ( {\sum }_{j ∈ V} {α}_{j}^{k} {W}_{DG}^{k} {h}_{j}^{L} ) \qquad ( 8 )$$

$${α}_{j} = \frac{exp ( e ( {s}_{t} , {h}_{j}^{L} ) )}{{\sum }_{k ∈ V} exp ( e ( {s}_{t} , {h}_{k}^{L} ) )} \qquad ( 9 )$$

Further, an embodiment calculates context vectors cₛ for chief complaint sequence P using the equations below:

$${c}_{s} = {s}_{t} + {‖}_{k = 1}^{K} ( {\sum }_{j ∈ \vert C \vert} {α}_{j}^{k} {W}_{DT}^{k} {p}_{j} ) \qquad ( 10 )$$

$${α}_{j} = \frac{exp ( e ( {s}_{t} , {p}_{j} ) )}{{\sum }_{k ∈ \vert C \vert} exp ( e ( {s}_{t} , {p}_{k} ) )} \qquad ( 11 )$$

Here, W_{DG} and W_{DT} are separate trainable decoder weights that differ query, key, value in the encoder.

To predict the next hidden state, an embodiment constructs the final context vector by concatenation cₜ=[c∥cₛ]. Such an embodiment then uses an input-feeding decoder where both sₜ and cₜ are passed as input to the calculate the next timestep hidden state sₜ₊₁. To predict the next word in abstract, the probability of each next token is calculated by scaling [sₜ∥cₜ] to the vocabulary size with another weight matrix and taking a softmax.

FIG. 6 is a simplified diagram of a decoder 648 that may be utilized in embodiments. The encoder 648 takes the graph 667, which may be the graph 567 from the encoder 545 and uses a graph transformer 660 and attention 661 (implemented using equations 8-11) to compute context vectors c_{g} 662 for the graph 667. The decoder 648 also takes the chief complaint 663 and processes the chief complaint 663 with the chief complaint encoder 664 and attention 661 to generate the context vector cₛ 665 for the chief complaint 663. The context vectors 662 and 665 are concatenated 666 to generate final context vector 666 cₜ.

The decoder 648 is input feeding and both previous timestep hidden state sₜ 669 and the context vector 666 are passed as input to calculate the next timestep hidden state 670. To generate the next word in the assessment/plan, the context vector cₜ 666 and sₜ 669 are concatenated 671 and then feed into a decoder architecture, such as LSTM or Transformer Decoder, to generate the probability of the next word among all words in predefined vocabulary. Since words in chief complaint 663 usually occur in the assessment/plan, an embodiment uses a copy mechanism 672 to increase the probability among words in chief complaint. After the final vocabulary distribution is obtained, the decoder 648 uses a softmax distribution 673 to get the highest word probability and uses this as the next word wₜ 674 in the assessment/plan being generated.

Another embodiment uses a decoder configured to generate an assessment or plan using all of the data described herein, e.g., the EHR data 441, similar patient data 442, medical knowledge data 443, and patient prior history data 444. In order to generate an accurate assessment or plan, such an embodiment first unifies the patient representations based on the graphs discussed above. Specifically, such an embodiment concatenates the patient specific information graph embedding c_{g} with previous visit code embedding cₜ to obtain the final patient embedding r. This final patient embedding r will contain information from four different sources: patent specific information, external medical knowledge, similar patient assessment, and previous visit prediction.

To train the encoder and decoder end-to-end, an embodiment treats the final patient embedding r as a latent variable that is marginalized to get the seq2seq probability P(y|Gₛ) via a top-K approximation as shown in equation 12:

P(y|Gₛ)=Σ_{z ∈top-k(p}_{_{γ}}(*|Gₛ)) P_{γ}(z|Gₛ)Πᵢ^{N}P_{θ}(y|r,y_{1:i-1}) (12)

The decoder module P_{θ}(y|r,y_{1:i-1}) can be modelled using any pre-existing decoder module. An embodiment uses BART-large (Lewis et al., 2020) which is a pretrained seq2seq transformer with 400M parameters. BART-large has achieved the state-of-the-art results on a diverse set of generation tasks in the general NLP domain.

Meta Learning

Although the VHA cohort comprises a sizable amount of training data (1,658,035 patients), there are thousands of diseases and conditions including common diseases, such as diabetes, and a long tail of less common or rare diseases, such as Huntington's disease. In addition, there are millions of domain specific clinical concepts (Bodenreider, 2004). Although the knowledge enriched graph transformer model of embodiments may mitigate such data sparsity and domain divergence challenges, an embodiment may also implement a meta-learning framework to further improve the assessment and plan generation.

Recent studies have integrated graphs into meta-learning. Focused on node classification, Meta-GNN (Zhou et al., 2019) used gradient-based meta-learning with task sampling. Meta-GNN classifies an unseen label set by observing other label sets in the same graph. To learn an unseen graph from other graphs with the same label set, GFL (Yao et al., 2020) learned a transferable metric space between nodes and the prototype of each class with a graph autoencoder. Focused on the task of link prediction, Meta-Graph (Bose et al., 2020) used graph signature functions learned from other label sets across multiple graphs. These algorithms, however, require message passing on the entire graph, which reduces the scalability and their effectiveness on inductiveness. Further, these existing methods focus on the general graph domain problems like AMINER citation (Tang et al., 2008), reddit post link dataset (Hamilton et al., 2017), and protein-protein interaction (Zitnik and Leskovec, 2017), not the graph to text problem solved by embodiments.

In recent years, research has explored meta-learning on concept graphs with long-tail relation. Xiong et al. (2018) and Chen et al. (2019) used metric-based models like relation similarity function or relation meta learner. However, these methods only learn from a single graph from a specific domain, which is not applicable to datasets used herein where patients are represented as multiple subgraphs. G-META (Huang and Zitnik, 2020) extracted relevant local subgraphs first, and then applied GNN on each subgraph individually. G-META also applied meta gradients to learn transferable knowledge. This resulted in an efficient node classification and link prediction. However, G-META only focused on graph structure. There is no existing functionality that integrates graph-to-text applications into an end-to-end meta learning framework.

Embodiments provide an innovative meta-learning framework for graph-to-text applications. An embodiment clusters patients based on their disease domains. An embodiment clusters patients based on their similarity. The most commonly used similarity metrics are nearest neighbor, where patient's information (ICD codes) is matched with other patients' codes to identify similar patients. Such an embodiment identifies similar patients based on their disease similarity. In turn, the similar patients (a cluster) can be used in an embodiment for fine-tuning (domain adaptation or meta-learning).

When migrating to a new domain, embodiments quickly adjust using limited training data. To achieve this goal, an embodiment defines a model and meta parameters. Model parameters θ₀ include the parameters in the model, and meta parameters θ₁ include parameters in the transmission layers and the encoder. Meta parameters θ₁ can be tuned to reduce the domain divergence. The following learning policy is adopted to update the two parameters in an embodiment.

For the training, an embodiment uses disease domains D* of the patient. The data Di of each domain i is split into three parts, a training set Dₜᵢⁱ, a development set D_{dev}ⁱ and a test set Dₜₑⁱ. Model training samples a pair of domains {i, j}, and then uses Dₜᵣⁱ,D_{dev}ⁱ to update the model parameter θ₀. The meta parameter θ₁ is updated using D_{dev}^{j}. The meta parameters are used to fine tune embodiments for different disease domains. An embodiment follows Finn et al. (2017) for gradients update.

The process below summarizes the learning policy for training an embodiment using disease domains. The training procedure includes:

Dataset D = D⁰, D⁰, ..., Dⁿ⁻¹;
INSTANT Model: M(θ);
for Dⁱ, D^{j} ϵ D do

| | |
|---|---|
| \| | // Model training, update the INSTANT model |
| \| | θᵢ ← Loss(Dⁱₜᵣ, Dⁱ_{dev}; M(θ)) ; |
| \| | // Meta training, update the transmission layers and encoder |
| \| | θᵢ′ ← Loss(D^{j}_{dev}; M(θ)) ; |

end
θ ← Σⁿ⁻¹ᵢ₌₀Loss(Dⁱₜᵣ; M(θ)) ;
θ ← (D^{d}ₜᵣ, D^{d}_{dev}; M(θ)) ;

According to an embodiment, each training procedure involves two consecutive training processes: model training and meta training. Each iteration mimics a domain adaptation of an embodiment. The meta parameter learns to handle domain divergence. Using meta learning, embodiments can be readily adjusted to match data of new disease domains.

An embodiment explores different approaches to cluster diseases into different domains. First, diseases can be clustered using the Medical Subject Heading (MeSH) hierarchy (Nelson et al., 2001). MeSH descriptors are organized in 16 general categories including the C category of diseases. Each general category is further divided into subcategories. In embodiments, the disease category can be further divided into 24 disease subcategories including infections, neoplasms, and musculoskeletal diseases, amongst others, which can be further divided into sub-sub categories. Such an embodiment may utilize different levels of disease hierarchies and may also employ other approaches for disease similarities, including functional associations between disease related genes and semantic associations between diseases (Cheng et al., 2014).

### Experimental Results

Hereinbelow, various experiments and the results thereof are described which demonstrate the effectiveness and some of the benefits of embodiments.

### Datasets

These experiments relied on a corpus of 25,200 outpatient EHR notes from hospitals and medical centers, from which about 17,500, 7600, and 100 notes were randomly selected for training, development, and test sets, respectively. Additional background information is added within the graph used for the experiments.

TABLE 2

| | ESO | EAS |
|---|---|---|
| Vocab | 74 K | 39 K |
| Tokens | 9.8 M | 2.2 M |
| Avg Len | 392 | 89 |
| Entity Types | 40 | — |
| Avg Vert | 7.91 (+5.29) | — |
| Avg Edge | 4.02 (+2.62) | — |

Table 2 includes vocabulary size of document (Vocab), number of total document tokens (Tokens), average document length (Avg Len), number of unique entity types (Entity Types), average number of vertices (Avg Vert), average number of edges for (Avg Edge), the EHR subjective and objective part (ESO), and the EHR assessment part (EAS). The average vertices and edges of ESO is split into two parts. The first part represents data from the patient specific information graph and the second part represents data from the patient background information graph.

### Baselines

The evaluation considers whether injecting data, e.g., the background medical data, into the patient graph improves the assessment generation task. As such, baseline models do not include such additional data. Other baseline models are based on the text-to-text encoder-decoder framework. For this, a first baseline model is N2MAG (Hu et al., 2020). A second baseline model is a T5 model trained on the large open domain data (Raffel et al., 2019). To mitigate the domain divergence challenge, a test utilizes stronger baseline models built using the meta-learning framework described in the previous section. Finally, another type of strong baseline models is built on fine tuning. In this case, models are trained on the entire VHA dataset and are then fine tuned to the disease domains of the patient.

The results discussed below evaluate whether injecting external clinical knowledge, similar patient information, and a patient's prior history improve embodiments. In such an evaluation, a baseline model is implemented without those elements. The results compare an embodiment against several baselines.

In an evaluation, a graph model only includes patient specific information and the background medical knowledge graph is left out to test the need for it, this is referred to as (BASIC). Then, the results compare an embodiment with augmented attention-over-attention pointer-generator network (N2MAG model) from Hu et al. (2020). The below discussion also compares the result of BASIC with self-attention based architectures. An implementation utilizes a text to text vanilla transformer with 6 layers of encoder and decoder. To test the ability of the background medical knowledge graph, results also compare the result of EXT (an embodiment implemented to use the background medical knowledge graph) to a pretrained generation model on large corpus T5 (Raffel et al., 2019), where T5-Small is the encoder-decoder model with 6 layers each, and T5-Base is the encoder-decoder model with 12 layers each.

The results also use finetuned models on the employed dataset. In further evaluation, to mitigate the domain divergence challenge, a stronger baseline model is built using the meta-learning framework described above. Finally, another type of strong baseline model is built on fine tuning. In this case, models are trained on the entire VHA dataset and fine tuned to the disease domains of the patient.

### Implementation

To implement the embodiment used to generate the results, embodiments, were trained end-to-end with EHR chief complaint text and relevant graphs as input and corresponding assessments as targets. An implementation used SGD optimization with momentum (Qian, 1999) and the best learning rate is 0.05 and momentum is 0.9 with gradient clipping. Models were trained for 25 epochs with early stopping (Prechelt, 1998) based on the validation loss, with most models stopping at approximately 15 epochs. Each word is imbedded into 500 vectors and the same dimension is used on hidden state size. As for the graph encoder, a graph attention network (Velickovic et al., 2018) with 6 layers with 4 heads was used. To encode chief complaint text, a 2 layer BiLSTM was employed. To avoid penalizing repeatedly attending to the same locations, coverage loss weight was set to 0.5. During inference, beam search with a beam size of 4 and beam width of 6 was used to generate EHR assessments. To prevent overfitting, a dropout rate of 0.1 (Srivastava et al., 2014) is used. For each method, experiments were run for 4 trials with random weight initialization, and the best model is selected to do evaluation for each method. Repeated sentences were manually removed before evaluation. The experiment was carried out on 2 TITANX GPUs. Each model finished training within 12 hours. Further, the code and settings are publicly available at https://github.com/whaleloops/mcag

### Evaluation Metrics

One evaluation metric that is utilized is BLEU (Papineni et al., 2002). BLEU is an evaluation metric for text generation that measures the intersection of n-grams between the generated assessment and the gold assessment. A better generated assessment usually achieves a higher BLEU score, as it shares more n-grams with the gold assessment.

ROUGE (Lin, 2004) is another evaluation metric that is utilized. ROUGE is an evaluation metric for summarization, that also measures the intersection of n-grams between the generated assessment and the gold assessment. However, unlike BLEU, ROUGE focuses on the n-grams appearing in the machine generated assessment as a measure of recall instead of precision. A better generated assessment usually achieves a higher ROUGE score, as it shares more n-grams with the gold assessment.

Human evaluation was also utilized. While BLEU and other automatic metrics are objective metrics that can be applied to large-volume test sets, it is also desirable to ensure that embodiments operate according to human evaluator. The results below rely on 4 doctor experts to provide human evaluation. The human evaluators were asked to compare each generated assessment and gold assessment from four perspectives: (1) Sentence Fluency: Is the generated assessment semantically coherent and meaningful, (e.g. “get a flu shot” is good and “drink a flu shot” is bad)? (2) Keywords Coverage: Do the keywords match between assessment and background? (Is the patient male or female? Age same? Times of visit same?) (3) Clinical Accuracy: Is the generated assessment semantically reasonable compared to the given background? (4) Differential Discussion: Coverage of elements in assessment sufficient? (Does it contain Problem? Differential Diagnoses? Discussion? Care/Politeness to patient). The grading scale for each perspective was from 1 to 5.

### Results

Table 3 shows assessments generated used the embodiments described herein.

TABLE 3

| Model | Text |
|---|---|
| N2MAG | ASSESSMENT: The patient attends today's OBESITY CONSULTATION. |
| | She seems to have a good amount of past nutrition EDUCATION. |
| BASIC | ASSESSMENT: The patient attends today's nutrition CONSULTATION to |
| | ADDRESS her OBESITY issue. 1. She is doing better on all BLOOD |
| | SUGAR MANAGEMENT. 2. she is exercising many times a week. At this |
| | point, I do feel comfortable having her move WEIGHT LOSS next step in our |
| | program. |
| EXT | ASSESSMENT: The patient attends today's nutrition CONSULTATION to |
| | ADDRESS her struggle with OBESITY. She is doing better on BLOOD |
| | SUGAR MANAGEMENT and suggestions made by this provider. She has |
| | made a number of changes to her diet and lifestyle over the past few months. |
| | She is very engaged in our appointment today and asked appropriate |
| | EXERCISE questions to the education that was provided. We talked about |
| | using Saxenda as an alternative. At this point, I do believe that her |
| | HEMOGLOBIN Alc step DOWNWARD. |

Table 3 shows example assessments generated by different models. The input and gold assessment are shown in FIG. 1. BASIC represents an embodiment which generates the assessment from the patient specific information graph. EXT is an embodiment which generates the assessment from patient specific information graph and background information graph. Medical keywords selected from entities and relations in the graph are marked as bold in Table 3. N2MAG does not have graph, so MetaMap and some rules are used to find these medical keywords.

Table 4 shows the BLEU and ROUGE scores for the assessments generated using embodiments and Table 5 shows the mean human evaluation scores. Intuitively, the more the generated assessments resembles the gold assessments, the better the model is.

TABLE 4

| Model | BLEU-1 | BLEU-2 | BLEU-3 | BLEU-4 | ROUGE-L |
|---|---|---|---|---|---|
| N2MAG | 9.726 | 5.449 | 2.12 | 1.412 | 22.334 |
| Transformer | 27.053 | 16.761 | 11.488 | 8.457 | 20.613 |
| BASIC | 27.926 | 17.117 | 12.158 | 9.046 | 23.289 |
| T5 Small | 28.534 | 17.720 | 12.323 | 9.190 | 20.419 |
| T5 Base | 30.542 | 18.006 | 12.124 | 8.772 | 19.155 |
| EXT | 38.731 | 26.667 | 20.299 | 15.942 | 30.662 |

Table 4 shows automatic scores of generated assessments. In Table 4, Transformer is the vanilla transformer with 6 layers encoder-decoder. T5 Small uses the same architecture, but is pretrained on a large corpus and T5 Base doubles the number of layers. BASIC uses a 6 layer encoder decoder model which generates an assessment from a patient specific information graph. EXT is a model which generates an assessment from patient specific information graph and background information graph.

Table 5 shows human evaluation results of generated assessments.

TABLE 5

| | Sentence | Keyword | Clinical | Differential |
|---|---|---|---|---|
| Model | Fluence | Coverage | Accuracy | Discussion |
| N2MAG | 2.92 | 2.14 | 2.07 | 1.97 |
| BASIC | 3.31 (+0.39) | 2.31 (+0.17) | 2.10 (+0.03) | 2.35 (+0.38) |
| EXT | 3.48 (+0.17) | 2.73 (+0.42) | 3.13 (+1.03) | 3.08 (+0.73) |
| Human | 3.70 (+0.22) | 3.23 (+0.50) | 3.55 (+0.42) | 3.38 (+0.390) |

Table 5 shows the mean scores for each evaluation metric of 30 EHR notes. Scores improved the most in each category are bolded.

The experimental results show that BLEU scores and scores in human evaluation are generally consistent with each other. It can be seen that BLEU scores are fairly low, which is reasonable as there could be multiple ways to compose an assessment given the background of a patient.

The graph based model of embodiments leads to high precision. Compared to the graph transformer based models, the pointer generator is more susceptible to two sources of errors: (1) the pointer generator tends to generate a shorter assessment centered upon a fewer number of medical keywords and (2) the pointer generator also lacks the ability to select multiple keywords and expand upon these keywords.

As shown in Table 3, the result produced from the pointer-generator only contains 2 medical keywords in bold, while the result produced from BASIC contains 5. Within the test dataset, the average number of medical keywords extracted from N2MAG and BASIC is 3.4 and 7.1, respectively.

Recall that BLEU measures precision: how often the tokens in the machine generated assessment appear in the doctor reference assessment. ROUGE measures recall: how often the tokens in the doctor reference assessment appear in the machine generated assessment. Although the embodiment without graph enhancement (BASIC) has a much better BLEU score compared to pointer-generator results, the embodiment without graph enhancement does not improve a lot in ROUGE-L compared to pointer-generator results. This shows that pointer-generator works as a summarization model, and its ability is restricted in keyword selection. As a result, pointer-generator methods tend to generate shorter assessments, hence gaining a more favorable score on ROUGE-L (the gap between the pointer generator and graph is closer according to ROUGE-L). This is also proven in human evaluation as well. Embodiments without graph enhancement achieve a +0.03 point improvement in clinical accuracy, but +0.38 point improvement in differential discussion and +0.39 point improvement in sentence fluency. In comparison to the pointer generator model, the graph model shows more capability to include medical keywords and generate related discussions and differential diagnoses.

The BASIC implementation can also be compared to a non-pretrained text-to-text transformer model. While transformers can be seen as GNNs from an architecture perspective, the embodiments use keywords (graph) extracted from text as input, while the baseline transformer model uses more text as input. However, as shown in Table 3, the performance of the non-pretrained text-to-text transformer model is similar to BASIC which does not use external knowledge. This shows that the medical assessment generation task relies mostly on keywords, and more irrelevant input would not do better in this task.

Incorporating background medical graphs results in generating assessments that are in better agreement with experts. Among two graph based models, enhancing the graph by expanding relevant background entities with UMLS further improves the quality of the generated assessments. By comparing clinical keywords identified among the generated and gold assessment, the expanding technique implemented in embodiments can increase the clinical keyword overlap from 35% to 97%. Graph enhancements further significantly improves Clinical Accuracy by +1.03 and Differential Discussion by +0.73. However, sentence fluency is not improved as the model architecture is not altered. This shows the importance of expanding relevant background entities from a graph level in this task as more information is given.

Explicit knowledge graph outperforms implicit pre-trained model. Even though pre-trained language models are able to answer queries structured as “fill-in-the-blank” cloze statements, and Petroni et al. (2019) have shown that factual relational knowledge already presents within these pretrained models, Poerner et al. (2019), however, demonstrated that these pre-trained language models could only capture shallow information stored in the knowledge base, and incorporating BERT with entity embedding outperforms original BERT (Peters et al., 2019).

Here, results yield similar findings, but in a text generation task. With automatic evaluations shown in Table 4, the EXT model with graph enhancement outperforms pre-trained T5-Small, where the number of parameters is about the same. By doubling the number of layers, TS-Base only increases a little in BLEU, but decreases slightly in ROUGE-L compared with T5-Small. Both pre-trained models outperform the non-pre-trained vanilla transformer. This may indicate that pre-trained language models from general web corpuses contain only limited knowledge on a specific domain (i.e., medical). This shows that explicitly integrating a self-attention encoder with knowledge graph improves the quality of text assessments and plans generated by embodiments compared to the pre-trained language model.

The results also show that assessment generation is an arduous task. Even doctor written assessments get a medium score of about 3.5 in Table 5 instead of the full 5 points.

Embodiments provide a novel approach for the task of generating medical assessments and plans from patient specific medical information and also relevant background information. Embodiments adapt the graph transformer model to the assessment generation task and also address the lack of relevant background medical knowledge by enhancing the graph with additional information, i.e., relevant medical background information. The results show that the graph transformer embodiments outperform text pointer-generator models, even without the help of additional background medical knowledge. In addition, enhancing the graph with relevant medical knowledge further improves the quality of the generated text, e.g., assessment. Experiments also show the current Text-to-Text Transformer pretrained on a large corpus may learn limit medical domain-specific knowledge. Further, text generation quality improvements can be made by incorporating domain-specific knowledge graphs.

Embodiments may also switch some entities to other irrelevant and improper tokens to make the graph model more resilient to these noises. Further, many EHRs are follow-up EHRs that are based on one or more previous EHR. As such, an embodiment may expand EHRs in time by applying temporal graph models to incorporate temporal information.

Key innovations of embodiments include knowledge inference using both structured data (e.g., ICD codes, medications, and lab results) and unstructured EHR notes. Embodiments infer rich clinical knowledge from notes with a SOAP structure. In this case, the chief complaint and subjective evidence lead to objective measurements. Assessments can be inferred from both subjective and objective evidence, all of which lead to specific plans.

Embodiments can infer assessments and plans using external medical knowledge. An embodiment identifies clinical concepts and concept relations from a SOAP note and augments the concept graph with external medical knowledge to infer new knowledge for the assessment and plan. One such implementation uses the large biomedical knowledge resources, Unified Medical Language System (UMLS) and MedlinePlus, both developed by the National Library of Medicine (NLM), for external medical knowledge. As shown in FIG. 3, the augmented concept graph from the external medical knowledge resources helps infer “Saxenda” in the assessment. In addition, the assessment and plan can also be inferred from a patient's prior conditions (longitudinal EHRs) and from similar patients' clinical trajectories.

The assessments and plans determined by embodiments can be used to provide computer-assisted clinical decision support systems (CDSS). Embodiments can be used to provide checks and prompts to medical providers and to modify the care and treatment patients receive.

Advantages of embodiments are multi-fold. First, existing knowledge-based CDSS are limited to specific clinical subdomains. For example, (Shortliffe, 1974) is an expert system for diagnosing bacterial infections and prescribing treatment for bacterial infections. In contrast, embodiments are able to cover a broad range of disease categories and help patients with complex diseases. This is achieved by training embodiments on large longitudinal EHRs, which include patients with a broad range of diseases and patients with complex diseases. Moreover, unlike previous machine learning models which have little interpretability, embodiments provide outputs that summarize the reasons that lead to the assessments and treatment plans.

Moreover, machine learning based models have been developed to predict structured outputs, such as the International Classification of Diseases, Clinical Modification (ICDCM) codes (Miotto et al., 2016) and medications (Duan et al., 2020). In contrast, embodiments infer clinical diagnoses and treatment plans from patient's self-reported symptoms, clinical test results, and observations, and output clinical assessments and treatment plans in natural language. Automated assessment and treatment plan generation is a novel application.

Thirdly, previous methods in EHR related text generation are based on the text-to-text encoder-decoder framework. For example, Zhang et al. (2018) used a combination of extractive and abstractive techniques to summarize radiology reports. In contrast, embodiments utilize a graph transformer model for graph-to-text generation for EHRs. The advantages of graph transformer models used in embodiments include mitigating the data sparsity challenge and the ability to include external medical knowledge for knowledge inference, both of which are key steps towards building effective and clinically actionable CDSS systems. Existing methods lack knowledge inference as described herein.

The clinical domain is complex, with thousands of diseases and their complex combinations. Moreover, there are millions of clinical concepts or domain specific medical jargon (Bodenreider, 2004), which further compound the challenges in developing CDSS systems. Therefore, even with large EHRs with millions of patients, the data sparsity remains a significant challenge. The graph-transformer models in embodiments mitigate the aforementioned challenges.

Fourthly, the graph-transformer models in embodiments are highly innovative in computer science. The models inject external knowledge into the end-to-end graph-transformer models for knowledge inference. This has not been done in any of the previous work in graph-to-text generation (Koncel-Kedziorski et al., 2019; Zhu et al., 2019; Guo et al., 2019). This allows embodiments to use external clinical knowledge that includes not only existing knowledge resources, but also dynamical information such as patients' prior history and information from similar patients, which improves knowledge inference. Embodiments implement innovative methods and systems to capture both existing knowledge resources and the dynamical information from EHRs and to integrate them with the graph-transformer models for end-to-end training.

Embodiments provide a highly innovative meta-learning framework to further mitigate the data sparsity challenge. This has not been done in any of the existing graph-to-text applications.

### Computer Implementation

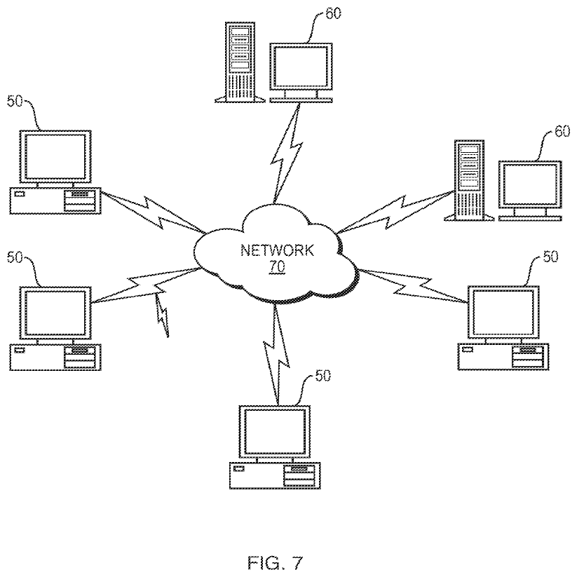

FIG. 7 illustrates a computer network or similar digital processing environment in which the above described graph-to-text frameworks and embodiments of the present invention may be implemented.

Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable.

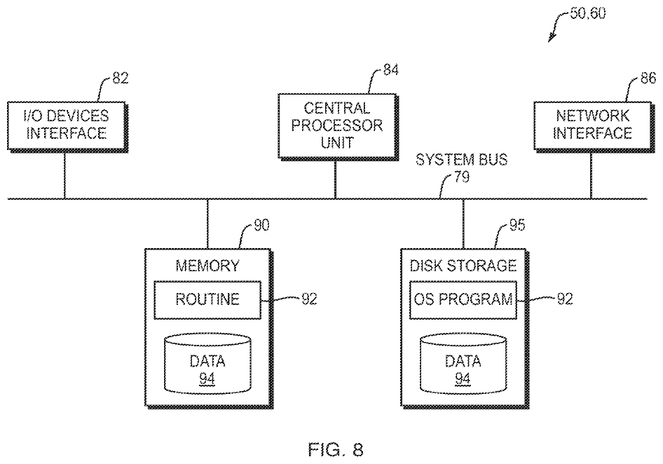

FIG. 8 is a diagram of the internal structure of a computer (e.g., client processor/device 50 or server computers 60) in the computer system of FIG. 7. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 7). Memory 90 provides volatile storage for computer software instructions 92 and data 94 used to implement embodiments of the present invention (e.g., code for implementing the method 220 or framework 440 described herein). Disk storage 95 provides non-volatile storage for computer software instructions 92 and data 94 used to implement embodiments of the present invention. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

In other embodiments, the program product 92 may be implemented as a so called Software as a Service (SaaS), or other installation or communication supporting end-users.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

### REFERENCES

Marilisa Amoia, Frank Diehl, Jesus Gimenez, Joe Pinto, Raphael Schumann, Fabian Stemmer, Paul Vozila, and Yi Zhang. 2018. Scalable wide and deep learning for computer assisted coding. In Proceedings of the 2018 Conference of the North American Chapter of the Association for Computational Linguistics: Human Language Technologies, Volume 3 (Industry Papers), pages 1-7, New Orleans-Louisiana. Association for Computational Linguistics.

Alan R. Aronson and Francois-Michel Lang. 2010. An overview of metamap: historical perspective and recent advances. Journal of the American Medical Informatics Association: JAMIA, 17 3:229-36.

Olivier Bodenreider. 2004. The Unified Medical Language System (UMLS): integrating biomedical terminology. Nucleic Acids Research, 32(supp11): D267-D270.

Edward Choi, Siddharth Biswal, Bradley Malin, Jon Duke, Walter F. Stewart, and Jimeng Sun. 2017. Generating multi-label discrete patient records using generative adversarial networks.

R. Scott Evans. 2017. Health records: Then, now, and in the future.

Jiaqi Guan, Runzhe Li, Sheng Yu, and Xuegong Zhang. 2018. Generation of synthetic electronic medical record text. 2018 IEEE International Conference on Bioinformatics and Biomedicine (BIBM).

Baotian Hu, Adarsha Bajracharya, and Hong Yu. 2020. Generating medical assessments using a neural network model: Algorithm development and validation. JMIR Med Inform, 8(1):e14971.

Rik Koncel-Kedziorski, Dhanush Bekal, Yi Luan, Mirella Lapata, and Hannaneh Hajishirzi. 2019. Text Generation from Knowledge Graphs with Graph Transformers. In Proceedings of the 2019 Conference of the North American Chapter of the Association for Computational Linguistics: Human Language Technologies, Volume 1 (Long and Short Papers), pages 2284-2293, Minneapolis, Minnesota. Association for Computational Linguistics.

Scott H. Lee. 2018. Natural language generation for electronic health records. npj Digital Medicine, 1(1).

Chin-Yew Lin. 2004. ROUGE: A package for automatic evaluation of summaries. In Text Summarization Branches Out, pages 74-81, Barcelona, Spain. Association for Computational Linguistics.

Kimberly J O'malley, Karon F. Cook, Matt D. Price, Kimberly Raiford Wildes, John F. Hurdle, and Carol M. Ashton. 2005. Measuring diagnoses: Icd code accuracy. Health services research, 40 5 Pt 2:1620-39.

Kishore Papineni, Salim Roukos, Todd Ward, and Wei-Jing Zhu. 2002. Bleu: a method for automatic evaluation of machine translation. In Proceedings of the 40th Annual Meeting of the Association for Computational Linguistics, pages 311-318, Philadelphia, Pennsylvania, USA. Association for Computational Linguistics.

Matthew E. Peters, Mark Neumann, Robert Logan, Roy Schwartz, Vidur Joshi, Sameer Singh, and Noah A. Smith. 2019. Knowledge enhanced contextual word representations. In Proceedings of the 2019 Conference on Empirical Methods in Natural Language Processing and the 9th International Joint Conference on Natural Language Processing (EMNLP-IJCNLP), pages 43-54, Hong Kong, China. Association for Computational Linguistics.

Fabio Petroni, Tim Rocktaschel, Sebastian Riedel, Patrick Lewis, Anton Bakhtin, Yuxiang Wu, and Alexander Miller. 2019. Language models as knowledge bases? In Proceedings of the 2019 Conference on Empirical Methods in Natural Language Processing and the 9th International Joint Conference on Natural Language Processing (EMNLP-IJCNLP), pages 2463-2473, Hong Kong, China. Association for Computational Linguistics.

Vivek Podder, Valerie Lew, and Sassan Ghassemzadeh. 2020. Soap notes. StatPearls Publishing.

Nina Poerner, Ulli Waltinger, and Hinrich Schütze. 2019. Bert is not a knowledge base (yet): Factual knowledge vs. name-based reasoning in unsupervised qa. ArXiv, abs/1911.03681.

Lutz Prechelt. 1998. Early stopping-but when? In Neural Networks: Tricks of the Trade, This Book is an Outgrowth of a 1996 NIPS Workshop, page 55-69, Berlin, Heidelberg. Springer-Verlag.

Ning Qian. 1999. On the momentum term in gradient descent learning algorithms. Neural Netw., 12(1):145-151.

Colin Raffel, Noam Shazeer, Adam Roberts, Katherine Lee, Sharan Narang, Michael Matena, Yanqi Zhou, Wei Li, and Peter J. Liu. 2019. Exploring the limits of transfer learning with a unified text-to-text transformer. arXiv e-prints.

Nitish Srivastava, Geoffrey Hinton, Alex Krizhevsky, Ilya Sutskever, and Ruslan Salakhutdinov. 2014. Dropout: A simple way to prevent neural networks from overfitting. J. Mach. Learn. Res., 15(1):1929-1958.

Gabriel Stanovsky, Julian Michael, Luke Zettlemoyer, and Ido Dagan. 2018. Supervised open information extraction. In Proceedings of the 2018 Conference of the North American Chapter of the Association for Computational Linguistics: Human Language Technologies, Volume 1 (Long Papers), pages 885-895, New Orleans, Louisiana. Association for Computational Linguistics.

Michael Subotin and Anthony Davis. 2014. A system for predicting ICD-10-PCS codes from electronic health records. In Proceedings of BioNLP 2014, pages 59-67, Baltimore, Maryland. Association for Computational Linguistics.

Ashish Vaswani, Noam Shazeer, Niki Parmar, Jakob Uszkoreit, Llion Jones, Aidan N Gomez, Lukasz Kaiser, and Illia Polosukhin. 2017. Attention is all you need. In I. Guyon, U. V. Luxburg, S. Bengio, H. Wallach, R. Fergus, S. Vishwanathan, and R. Garnett, editors, Advances in Neural Information Processing Systems 30, pages 5998-6008. Curran Associates, Inc.

Petar Velickovic, Guillem Cucurull, Arantxa Casanova, Adriana Romero, Pietro Lio, and Yoshua Bengio. 2018. Graph Attention Networks. International Conference on Learning Representations.

Sam Wiseman, Stuart Shieber, and Alexander Rush. 2017. Challenges in data-to-document generation. In Proceedings of the 2017 Conference on Empirical Methods in Natural Language Processing, pages 2253-2263, Copenhagen, Denmark. Association for Computational Linguistics.

2021. Department of Veterans Affairs. Veterans Health Administration: Providing Health Care for Veterans. https://www.va.gov/health/ (accessed 29 January 2021).

I. D. Adams, M. Chan, P. C. Clifford, W. M. Cooke, V. Dallos, F. T. De Dombal, M. H. Edwards, D. M. Hancock, D. J. Hewett, and N. McIntyre. 1986. Computer aided diagnosis of acute abdominal pain: a multicentre study. Br Med J (Clin Res Ed), 293(6550):800-804. Number: 6550 Publisher: British Medical Journal Publishing Group.

A. R Aronson. 2001. Effective mapping of biomedical text to the UMLS Metathesaurus: the MetaMap program. Proc AMIA Symp, pages 17-21.

G. O. Barnett, J. J. Cimino, J. A. Hupp, and E. P. Hoffer. 1987. DXplain. An evolving diagnostic decision- support system. JAMA, 258(1):67-74. Number: 1.

G. R Bergus, C. S Randall, S. D Sinift, and D. M Rosenthal. 2000. Does the structure of clinical questions affect the outcome of curbside consultations with specialty colleagues? Arch Fam Med, 9(6):541-7. Number: 6 1063-3987 Journal Article.

Chandra Bhagavatula, Ronan Le Bras, Chaitanya Malaviya, Keisuke Sakaguchi, Ari Holtzman, Hannah Rashkin, Doug Downey, Wen tau Yih, and Yejin Choi. 2020. Abductive commonsense reasoning. In International Conference on Learning Representations.

Olivier Bodenreider. 2004. The Unified Medical Language System (UMLS): integrating biomedical terminology. Nucleic Acids Research, 32(supp11): D267-D270.

Avishek Joey Bose, Ankit Jain, Piero Molino, and William L. Hamilton. 2020. Meta-graph: Few shot link prediction via meta learning.

Tom B. Brown, Benjamin Mann, Nick Ryder, Melanie Subbiah, Jared Kaplan, Prafulla Dhariwal, Arvind Nee- lakantan, Pranav Shyam, Girish Sastry, Amanda Askell, Sandhini Agarwal, Ariel Herbert-Voss, Gretchen Krueger, Tom Henighan, Rewon Child, Aditya Ramesh, Daniel M. Ziegler, Jeffrey Wu, Clemens Winter, Christopher Hesse, Mark Chen, Eric Sigler, Mateusz Litwin, Scott Gray, Benjamin Chess, Jack Clark, Christopher Berner, Sam McCandlish, Alec Radford, Ilya Sutskever, and Dario Amodei. 2020. Language models are few shot learners.

Yonggang Cao, Feifan Liu, Pippa Simpson, Lamont Antieau, Andrew Bennett, James J Cimino, John Ely, and Hong Yu. 2011. AskHERMES: An online question answering system for complex clinical questions. Journal of Biomedical Informatics, 44(2):277-288. Number: 2.

Subhagata Chattopadhyay, Suvendu Banerjee, Fethi A. Rabhi, and U. Rajendra Acharya. 2013. A Case- Based Reasoning system for complex medical diagnosis. Expert Systems, 30(1):12-20. Number: 1 Pub-lisher: Wiley Online Library.

Jinying Chen, Emily Druhl, Balaji Polepalli Ramesh, Thomas K Houston, Cynthia A Brandt, Donna M Zulman, Varsha G Vimalananda, Samir Malkani, and Hong Yu. 2018. A natural language processing system that links medical terms in electronic health record notes to lay definitions: system development using physician reviews. Journal of medical Internet research, 20(1):e26.

Mingyang Chen, Wen Zhang, Wei Zhang, Qiang Chen, and Huajun Chen. 2019. Meta relational learning for few-shot link prediction in knowledge graphs. In Proceedings of the 2019 Conference on Empirical Methods in Natural Language Processing and the 9th International Joint Conference on Natural Language Processing (EMNLP-IJCNLP), pages 4217-4226, Hong Kong, China. Association for Computational Linguistics.

Liang Cheng, Jie Li, Peng Ju, Jiajie Peng, and Yadong Wang. 2014. SemFunSim: A New Method for Measuring Disease Similarity by Integrating Semantic and Gene Functional Association. PLOS ONE, 9(6):e99415. Number: 6 Publisher: Public Library of Science.

Edward Choi, Mohammad Taha Bahadori, and Jimeng Sun. 2015. Doctor AI: Predicting Clinical Events via Recurrent Neural Networks. arXiv:1511.05942 [cs]. ArXiv: 1511.05942.

Edward Choi, Mohammad Taha Bahadori, Jimeng Sun, Joshua Kulas, Andy Schuetz, and Walter Stewart. 2016. Retain: An interpretable predictive model for healthcare using reverse time attention mechanism. In Advances in Neural Information Processing Systems, volume 29, pages 3504-3512. Curran Associates, Inc.

J. Cimino. 2008. Infobuttons: anticipatory passive decision support. AMIA Annu Symp Proc, pages 1203-4. 1559-4076 (Electronic) Journal Article.

J. J. Cimino. 1994. Controlled medical vocabulary construction: Methods from the canon group. JAMIA, 1:296-7.

J. J Cimino. 2000. From data to knowledge through concept-oriented terminologies: experience with the Medical Entities Dictionary. J Am Med Inform Assoc, 7(3):288-97. Number: 3 1067-5027 Journal Article.

J. J Cimino. 2006. Use, usability, usefulness, and impact of an infobutton manager. AMIA Annu Symp Proc, pages 151-5. 1559-4076 (Electronic) Journal Article.

J. J Cimino and D. V Borotsov. 2008. Leading a horse to water: using automated reminders to increase use of online decision support. AMIA Annu Symp Proc, pages 116-20. 1942-597X (Electronic) Journal Article Research Support, U.S. Gov't, P.H.S.

J. J Cimino, J. Li, M. Allen, L. M Currie, M. Graham, V. Janetzki, N. J Lee, S. Bakken, and V. L Patel. 2004. Practical considerations for exploiting the world wide web to create infobuttons. Medinfo, 11(Pt 1):277-81. Number: Pt 1.

James J. Cimino, Tiffani J. Bright, and Jianhua Li. 2007. Medication reconciliation using natural language processing and controlled terminologies. Studies in Health Technology and Informatics, 129(Pt 1):679-683. Number: Pt 1.

F. T. De Dombal, D. J. Leaper, John R. Staniland, A. P. McCann, and Jane C. Horrocks. 1972. Computer-aided diagnosis of acute abdominal pain. Br Med J, 2(5804):9-13. Number: 5804 Publisher: British Medical Journal Publishing Group.

Kevin Donnelly. 2006. SNOMED-CT: The advanced terminology and coding system for eHealth. Studies in health technology and informatics, 121:279. Publisher: IOS Press; 1999.

H. Duan, Z. Sun, W. Dong, K. He, and Z. Huang. 2020. On Clinical Event Prediction in Patient Treatment Trajectory Using Longitudinal Electronic Health Records. IEEE Journal of Biomedical and Health Informatics, 24(7):2053-2063. Number: 7 Conference Name: IEEE Journal of Biomedical and Health Informatics.

J. W Ely, R. J Burch, and D. C Vinson. 1992. The information needs of family physicians: case-specific clinical questions. J Fam Pract, 35(3):265-9. Number: 3 0094-3509 (Print) Comparative Study Journal Article Research Support, U.S. Gov't, P.H.S.

J. W Ely, J. A Osheroff, M. L Chambliss, M. H Ebell, and M. E Rosenbaum. 2005. Answering physicians' clinical questions: obstacles and potential solutions. J Am Med Inform Assoc, 12(2):217-24. Number: 2 1067-5027 Journal Article.

J. W Ely, J. A Osheroff, M. H Ebell, G. R Bergus, B. T Levy, M. L Chambliss, and E. R Evans. 1999. Analysis of questions asked by family doctors regarding patient care. BMJ, 319(7206):358-61. Number: 7206 0959-8138 Journal Article.

J. W Ely, J. A Osheroff, M. H Ebell, M. L Chambliss, D. C Vinson, J. J Stevermer, and E. A Pifer. 2002. Obstacles to answering doctors' questions about patient care with evidence: qualitative study. Bmj, 324(7339):710. Number: 7339 1468-5833 Journal Article.

Chelsea Finn, Pieter Abbeel, and Sergey Levine. 2017. Model-agnostic meta-learning for fast adaptation of deep networks. In International Conference on Machine Learning, pages 1126-1135. PMLR.

Reed M. Gardner, Bette B. Maack, R. Scott Evans, and Stanley M. Huff. 1992. Computerized medical care: the HELP system at LDS Hospital. J AHIMA, 63(6):68-78. Number: 6.

Mary K. Goldstein. 2013. IMPLEMENTING REAL-TIME CLINICAL DECISION SUPPORT FOR HEALTH PROFESSIONALS WITHIN WORKFLOW: ATHENA-CDS. In ANNALS OF BEHAVIORAL MEDICINE, volume 45, pages S62-S62. SPRINGER 233 SPRING ST, NEW YORK, NY 10013 USA.

Jiatao Gu, Zhengdong Lu, Hang Li, and Victor OK Li. 2016. Incorporating copying mechanism in sequence-to-sequence learning. arXiv preprint arXiv:1603.06393.

Jian Guan, Fei Huang, Zhihao Zhao, Xiaoyan Zhu, and Minlie Huang. 2020. A knowledge-enhanced pretraining model for commonsense story generation.

Jiaqi Guan, Runzhe Li, Sheng Yu, and Xuegong Zhang. 2018. Generation of synthetic electronic medical record text. In 2018 IEEE International Conference on Bioinformatics and Biomedicine (BIBM), pages 374-380. IEEE.

Zhijiang Guo, Yan Zhang, Zhiyang Teng, and Wei Lu. 2019. Densely connected graph convolutional networks for graph-to-sequence learning. Transactions of the Association for Computational Linguistics, 7:297-312.

Will Hamilton, Zhitao Ying, and Jure Leskovec. 2017. Inductive representation learning on large graphs. In Advances in Neural Information Processing Systems, volume 30, pages 1024-1034. Curran Associates, Inc.

Baotian Hu, Adarsha Bajracharya, and Hong Yu. 2020. Generating Medical Assessments Using a Neural Network Model: Algorithm Development and Validation. JMIR Medical Informatics, 8(1):e14971. Number: 1 Company: MIR Medical Informatics Distributor: JMIR Medical Informatics Institution: JMIR Medical Informatics Label: JMIR Medical Informatics Publisher: JMIR Publications Inc., Toronto, Canada.

Kexin Huang and Marinka Zitnik. 2020. Graph meta learning via local subgraphs. NeurIPS.

A. Ittycheriah, M. Franz, and S. Roukos. 2001. IBM's statistical question answering system—TREC-10. In TREC 2001, pages 644-652.

Abhyuday Jagannatha, Feifan Liu, Weisong Liu, and Hong Yu. 2019. Overview of the First Natural Language Processing Challenge for Extracting Medication, Indication, and Adverse Drug Events from Electronic Health Record Notes (MADE 1.0). Drug Safety, (1):99-111.

Abhyuday Jagannatha and Hong Yu. 2016a. Bidirectional Recurrent Neural Networks for Medical Event Detection in Electronic Health Records. arXiv:1606.07953 [cs]. ArXiv: 1606.07953.

Abhyuday N. Jagannatha and Hong Yu. 2016b. Structured prediction models for RNN based sequence labeling in clinical text. In Proceedings of the Conference on Empirical Methods in Natural Language Processing, volume 2016, pages 856-865.

Samay Jain, Steven E. Lo, and Elan D. Louis. 2006. Common Misdiagnosis of a Common Neurological Disorder: How Are We Misdiagnosing Essential Tremor? Archives of Neurology, 63(8):1100-1104. Number: 8.

Ashish K. Jha, Catherine M. DesRoches, Eric G. Campbell, Karen Donelan, Sowmya R. Rao, Timothy G. Ferris, Alexandra Shields, Sara Rosenbaum, and David Blumenthal. 2009. Use of Electronic Health Records in U.S. Hospitals. New England Journal of Medicine, 360(16):1628-1638. Number: 16 Publisher: Massachusetts Medical Society eprint: https://doi.org/10.1056/NEJMsa0900592.

Haozhe Ji, Pei Ke, Shaohan Huang, Furu Wei, Xiaoyan Zhu, and Minlie Huang. 2020. Language Generation with Multi-Hop Reasoning on Commonsense Knowledge Graph. In Proceedings of the 2020 Conference on Empirical Methods in Natural Language Processing, pages 725-736. ArXiv:2009.11692v1.

Alok Kapoor, Azraa Amroze, Jessica Golden, Sybil Crawford, Kevin O′Day, Rasha Elhag, Ahmed Nagy, Steve A. Lubitz, Jane S. Saczynski, Jomol Mathew, and David D. McManus. 2018. SUPPORT Piloting a Multi. Journal of the American Heart Association: Cardiovascular and Cerebrovascular Disease, 7(17). Number: 17.

Rik Koncel-Kedziorski, Dhanush Bekal, Yi Luan, Mirella Lapata, and Hannaneh Hajishirzi. 2019. Text Generation from Knowledge Graphs with Graph Transformers. In Proceedings of the 2019 Conference of the North American Chapter of the Association for Computational Linguistics: Human Language Technologies, Volume 1 (Long and Short Papers), pages 2284-2293, Minneapolis, Minnesota. Association for Computational Linguistics.

Scott H. Lee. 2018. Natural language generation for electronic health records. NPJ digital medicine, 1(1):1-7. Number: 1 Publisher: Nature Publishing Group.

Mike Lewis, Yinhan Liu, Naman Goyal, Marjan Ghazvininejad, Abdelrahman Mohamed, Omer Levy, Veselin Stoyanov, and Luke Zettlemoyer. 2020. BART: Denoising sequence-to-sequence pre-training for natural language generation, translation, and comprehension. In Proceedings of the 58th Annual Meeting of the Association for Computational Linguistics, pages 7871-7880, Online. Association for Computational Linguistics.

Fei Li and Hong Yu. 2020. ICD Coding from Clinical Text Using Multi-Filter Residual Convolutional Neural Network. Proceedings of the AAAI Conference on Artificial Intelligence, 34(05):8180-8187. Number: 05.

Yikuan Li, Shishir Rao, Jose Roberto Ayala Solares, Abdelaali Hassaine, Rema Ramakrishnan, Dexter Canoy, Yajie Zhu, Kazem Rahimi, and Gholamreza Salimi-Khorshidi. 2020. BEHRT: Transformer for Electronic Health Records. Scientific Reports, 10(1):7155.

Chin-Yew Lin. 2004. ROUGE: A package for automatic evaluation of summaries. In Text Summarization Branches Out, pages 74-81, Barcelona, Spain. Association for Computational Linguistics.

Gary Marcus. 2020. The Next Decade in AI: Four Steps Towards Robust Artificial Intelligence. Technical report. ArXiv:2002.06177v3.

Joshua Maynez, Shashi Narayan, Bernd Bohnet, and Ryan McDonald. 2020. On Faithfulness and Factuality in Abstractive Summarization. In The 58th Annual Meeting of the Association for Computational Linguistics. ArXiv:2005.00661v1.

Matthew Menear, Marc-André Blanchette, Olivier Demers-Payette, and Denis Roy. 2019. A framework for value-creating learning health systems. Health Research Policy and Systems, 17(1):79. Number: 1.

Naomi Miller, Eve-Marie Lacroix, and Joyce EB Backus. 2000. Medlineplus: building and maintaining the national library of medicine's consumer health web service. Bulletin of the Medical Library Association, 88(1):11.

Riccardo Miotto, Li, Brian A. Kidd, and Joel T. Dudley. 2016. Deep Patient: An Unsupervised Representation to Predict the Future of Patients from the Electronic Health Records. Scientific Reports, 6.

Stuart J. Nelson, W. Douglas Johnston, and Betsy L. Humphreys. 2001. Relationships in medical subject headings (MeSH). In Relationships in the Organization of Knowledge, pages 171-184. Springer.

Kishore Papineni, Salim Roukos, Todd Ward, and Wei-Jing Zhu. 2002. Bleu: a method for automatic evaluation of machine translation. In Proceedings of the 40th Annual Meeting of the Association for Computational Linguistics, pages 311-318, Philadelphia, Pennsylvania, USA. Association for Computational Linguistics.

Fabio Petroni, Tim Rocktäschel, Sebastian Riedel, Patrick Lewis, Anton Bakhtin, Yuxiang Wu, and Alexander Miller. 2019. Language models as knowledge bases? In Proceedings of the 2019 Conference on Empirical Methods in Natural Language Processing and the 9th International Joint Conference on Natural Language Processing (EMNLP-IJCNLP), pages 2463-2473, Hong Kong, China. Association for Computational Linguistics.

Eliseo J. Perez-Stable, Jeanne Miranda, Ricardo F. Muñoz, and Yu-Wen Ying. 1990. Depression in medical outpatients: under recognition and misdiagnosis. Archives of Internal Medicine, 150(5):1083-1088. Number: 5 Publisher: American Medical Association.

Alec Radford, Karthik Narasimhan, Tim Salimans, and Ilya Sutskever. 2018. Improving language understanding by generative pre-training.

Colin Raffel, Noam Shazeer, Adam Roberts, Katherine Lee, Sharan Narang, Michael Matena, Yanqi Zhou, Wei Li, and Peter J. Liu. 2019. Exploring the limits of transfer learning with a unified text-to-text transformer. arXiv e-prints.

Bhanu P Rawat, Fei Li, and Hong Yu. 2019. Naranjo Question Answering using End-to-End Multi-task Learning Model. 25th ACM SIGKDD Conference on Knowledge Discovery and Data Mining (KDD), pages 2547— 2555.

Alexander M. Rush, Sumit Chopra, and Jason Weston. 2015. A neural attention model for abstractive sentence summarization. In Proceedings of the 2015 Conference on Empirical Methods in Natural Language Processing, pages 379-389, Lisbon, Portugal. Association for Computational Linguistics.

Abigail See, Peter J. Liu, and Christopher D. Manning. 2017. Get to the point: Summarization with pointer- generator networks. arXiv preprint arXiv:1704.04368.

Edward H. Shortliffe and James J. Cimino. 2014. Biomedical informatics: computer applications in health care and biomedicine. Springer.

Edward Hance Shortliffe. 1974. MYCIN: a rule-based computer program for advising physicians regarding antimicrobial therapy selection. Technical report, STANFORD UNIV CALIF DEPT OF COMPUTER SCIENCE.

Gabriel Stanovsky, Julian Michael, Luke Zettlemoyer, and Ido Dagan. 2018. Supervised open information extraction. In Proceedings of the 2018 Conference of the North American Chapter of the Association for Computational Linguistics: Human Language Technologies, Volume 1 (Long Papers), pages 885-895, New Orleans, Louisiana. Association for Computational Linguistics.

Jie Tang, Jing Zhang, Limin Yao, Juanzi Li, Li Zhang, and Zhong Su. 2008. Arnetminer: Extraction and mining of academic social networks. In Proceedings of the 14th ACM SIGKDD International Conference on Knowledge Discovery and Data Mining, KDD '08, page 990-998, New York, NY, USA. Association for Computing Machinery.

T. Timpka and E. Arborelius. 1990. The GP's dilemmas: a study of knowledge need and use during health care consultations. Methods Inf Med, 29(1):23-9. Number: 1 0026-1270 (Print) Journal Article Research Support, Non-U.S. Gov't.

Zhaopeng Tu, Zhengdong Lu, Yang Liu, Xiaohua Liu, and Hang Li. 2016. Modeling coverage for neural machine translation. arXiv preprint arXiv:1601.04811.

Ashish Vaswani, Noam Shazeer, Niki Parmar, Jakob Uszkoreit, Llion Jones, Aidan N Gomez, L ukasz Kaiser, and Illia Polosukhin. 2017. Attention is all you need. In I. Guyon, U. V. Luxburg, S. Bengio, H. Wallach, R. Fergus, S. Vishwanathan, and R. Garnett, editors, Advances in Neural Information Processing Systems 30, pages 5998-6008. Curran Associates, Inc.

Oriol Vinyals, Meire Fortunato, and Navdeep Jaitly. 2017. Pointer Networks. arXiv:1506.03134 [cs, stat]. ArXiv: 1506.03134.

Lawrence L. Weed. 1968. Medical records that guide and teach. New England Journal of Medicine, 278(12):652-657. Number: 12 Publisher: Mass Medical Soc.

Wenhan Xiong, Mo Yu, Shiyu Chang, Xiaoxiao Guo, and William Yang Wang. 2018. One-shot relational learning for knowledge graphs. In Proceedings of the 2018 Conference on Empirical Methods in Natural Language Processing, pages 1980-1990, Brussels, Belgium. Association for Computational Linguistics.

Zhichao Yang and Hong Yu. 2020. Generating Accurate Electronic Health Assessment from Medical Graph. In Findings of the Association for Computational Linguistics: EMNLP 2020, pages 3764-3773, Online. Association for Computational Linguistics.

Huaxiu Yao, Chuxu Zhang, Ying Wei, Meng Jiang, Suhang Wang, Junzhou Huang, Nitesh Chawla, and Zhen- hui Li. 2020. Graph few-shot learning via knowledge transfer. Proceedings of the AAAI Conference on Artificial Intelligence, 34(04):6656-6663.

Hong Yu and David Kaufman. 2007. A cognitive evaluation of four online search engines for answering definitional questions posed by physicians. In Biocomputing 2007, pages 328-339. World Scientific.

Hyeong Won Yu, Maqbool Hussain, Muhammad Afzal, Taqdir Ali, June Young Choi, Ho-Seong Han, and Sungyoung Lee. 2019. Use of mind maps and iterative decision trees to develop a guideline-based clinical decision support system for routine surgical practice: case study in thyroid nodules. Journal of the American Medical Informatics Association, 26(6):524-536. Number: 6 Publisher: Oxford Academic.

Jinghe Zhang, Kamran Kowsari, J. Harrison, Jennifer M. Lobo, and L. Barnes. 2018a. Patient2vec: A personalized interpretable deep representation of the longitudinal electronic health record. IEEE Access, 6:65333-65346.

Yuhao Zhang, Daisy Yi Ding, Tianpei Qian, Christopher D. Manning, and Curtis P. Langlotz. 2018b. Learning to summarize radiology findings.

Fan Zhou, Chengtai Cao, Kunpeng Zhang, Goce Trajcevski, Ting Zhong, and Ji Geng. 2019. Meta-gnn: On few-shot node classification in graph meta-learning. In Proceedings of the 28th ACM International Conference on Information and Knowledge Management, CIKM '19, page 2357-2360, New York, NY, USA. Association for Computing Machinery.

Jie Zhu, Junhui Li, Muhua Zhu, Longhua Qian, Min Zhang, and Guodong Zhou. 2019. Modeling graph structure in transformer for better AMR-to-text generation. In Proceedings of the 2019 Conference on Empirical Methods in Natural Language Processing and the 9th International Joint Conference on Natural Language Processing (EMNLP-IJCNLP), pages 5459-5468, Hong Kong, China. Association for Computational Linguistics.

Marinka Zitnik and Jure Leskovec. 2017. Predicting multicellular function through multi-layer tissue networks. Bioinformatics, 33(14):i190-i198.

Wang et al., “Knowledge Graph Embedding by Translating on Hyperplanes,” available at https://ojs.aaai.org/index.php/AAAI/article/view/8870.

Bansal et al., “A2N: Attending to Neighbor for Knowledge Graph Inference,” “Proceeding of the 57^{th} Annual Meeting of the Association for Computational Linguistics,” pages 4387-4392, Jul. 28, 2019.

Shang et al., “Pre-training of Graph Augmented Transformers for Medication Recommendation,” Proceedings of the Twenty-Eight International Joint Conference on Artificial Intelligence, pages 5953-5959.

What is claimed is:

1. A computer-implemented method of generating medical support text based on patient medical data, the method comprising:
receiving medical data for a given patient;
generating a patient knowledge graph for the given patient based on the received medical data;
generating an expanded graph by expanding the generated patient knowledge graph based upon supplementary data; and
generating medical support text for the given patient based upon the expanded graph.

2. The method of the claim 1 wherein the received medical data includes text describing medical symptoms for the given patient.

3. The method of claim 1 wherein receiving the medical data comprises:
accessing an EHR database;
obtaining an EHR for the given patient from the accessed EHR database, wherein the obtained EHR comprises the medical data for the given patient.

4. The method of claim 3 wherein the obtained EHR is structured to include: a chief complaint, subjective data regarding the given patient, objective data regarding the given patient, an assessment of the given patient, and a treatment plan for the given patient.

5. The method of claim 1 wherein generating a patient knowledge graph based on the received medical data comprises:
natural language processing the received medical data.

6. The method of claim 5 wherein natural language processing the received medical data comprises:
extracting concept-relation-concept triples from the received medical data.

7. The method of claim 1 wherein the patient knowledge graph is a graph indicating relations between concepts in the received medical data.

8. The method of claim 1 wherein the supplementary data is a concept graph.

9. The method of claim 8 wherein expanding the generated patient knowledge graph based upon the supplementary data comprises:
computing a graph union of the patient knowledge graph and the concept graph, wherein the computed graph union is the expanded graph.

10. The method of claim 8 wherein the concept graph is an external medical knowledge concept graph.

11. The method of claim 1 wherein expanding the generated patient knowledge graph comprises:
performing a maximum inner product search (MIPS) of a patient database to identify one or more patients similar to the given patient;
obtaining medical data for the identified one or more patients similar to the given patient; and
expanding the generated patient knowledge graph using the obtained medical data for the identified one or more patients similar to the given patient.

12. The method of claim 11 wherein the obtained medical data for the identified one or more patients comprises at least one of: diagnoses ICD codes for the identified one or more patients; assessments for the identified one or more patients; and treatment plans for the one or more patients.

13. The method of claim 1 wherein expanding the generated patient knowledge graph comprises:
obtaining lab, diagnosis, and medication codes for one or more previous medical appointments for the given patient;
predicting at least one of a medication code and a diagnosis code for a future medical appointment for the given patient based on the obtained lab, diagnosis, and medication codes for one or more previous medical appointments for the given patient; and
expanding the generated patient knowledge graph based upon the predicted at least one medication code and diagnosis code for the future medical appointment.

14. The method of claim 1 wherein the support text is at least one of:
a medical assessment for the given patient; and
a treatment plan for the given patient.

15. The method of claim 1 wherein the support text is natural language text.

16. A system for generating medical support text based on patient medical data, the system comprising:
a processor; and
a memory with computer code instructions stored thereon, the processor and the memory, with the computer code instructions, being configured to cause the system to:
receive medical data for a given patient;
generate a patient knowledge graph for the given patient based on the received medical data;
generate an expanded graph by expanding the generated patient knowledge graph based upon supplementary data; and
generate medical support text for the given patient based upon the expanded graph.

17. The system of claim 16 wherein, in generating the knowledge graph for the given patient based on the received medical data, the processor and the memory, with the computer code instructions, are further configured to cause the system to:
perform natural language processing on the received medical data to extract concept-relation-concept triples from the received medical data.

18. The system of claim 16 wherein the supplementary data is an external medical knowledge concept graph and where, in expanding the generated patient knowledge graph based upon the supplementary data, the processor and the memory, with computer code instructions, are configured to cause the system to:
compute a graph union of the patient knowledge graph and the external medical knowledge concept graph, wherein the computed graph union is the expanded graph.

19. The system of claim 16 wherein, in expanding the generated patient knowledge graph based upon the supplementary data, the processor and the memory, with the computer code instructions, are configured to cause the system to perform at least one of:
(i) performing a maximum inner product search (MIPS) of a patient database to identify one or more patients similar to the given patient, obtaining medical data for the identified one or more patients similar to the given patient, and expanding the generated patient knowledge graph using the obtained medical data for the identified one or more patients similar to the given patient; and
(ii) obtaining lab, diagnosis, and medication codes for one or more previous medical appointments for the given patient, predicting at least one of a medication code and a diagnosis code for a future medical appointment for the given patient based on the obtained lab, diagnosis, and medication codes for one or more previous medical appointments for the given patient, expanding the generated patient knowledge graph based upon the predicted at least one medication code and diagnosis code for the future medical appointment.

20. A computer program product for generating medical support text based on patient medical data, the computer program product comprising:
one or more non-transitory computer-readable storage devices and program instructions stored on at least one of the one or more storage devices, the program instructions, when loaded and executed by a processor, cause an apparatus associated with the processor to:
receive medical data for a given patient;
generate a patient knowledge graph for the given patient based on the received medical data;
generate an expanded graph by expanding the generated patient knowledge graph based upon supplementary data; and
generate medical support text for the given patient based upon the expanded graph.
